(19) United States
(12) Patent Application Publication
(10) Pub. No.: US 2026/0240744 A1
(43) Pub. Date: Aug. 20, 2026

(54) Low-Specific-Gravity Whipped Hair Mask Featuring Hydrolyzed Vegetable Protein and Silybum Marianum for Disulfide Bond Restoration, Split-End Visual Enhancement, and Rinse-Off Convenience

(71) Applicant: Divi Official LLC, Irving, TX (US)

(72) Inventors: Erica Ashley Hendricks, McKinney, TX (US); Rachael Carolena Fox, Coppell, TX (US)

(21) Appl. No.: 19/058,349

(22) Filed: Feb. 20, 2025

(51) Int. Cl. A61K 8/64; A61K 8/58; A61K 8/73; A61K 8/898; A61K 8/9789; A61Q 5/00

(52) U.S. Cl. CPC A61K 8/645; A61K 8/585; A61K 8/735; A61K 8/898; A61K 8/9789; A61Q 5/002; A61K 2800/30; A61K 2800/522; A61K 2800/57; A61K 2800/70

(57) ABSTRACT

A rinse-off hair treatment composition and method is disclosed, comprising a hydrolyzed vegetable protein-silicone hybrid including Hydrolyzed Vegetable Protein PG-Propyl Silanetriol to restore disulfide bonds and reinforce keratin structures, and a botanical antioxidant including Silybum Marianum Seed Extract to neutralize oxidative stress and protect repaired bonds. The composition further includes secondary active ingredients, including Sodium Hyaluronate, Hydroxypropyltrimonium Hyaluronate, and Hydrolyzed Sodium Hyaluronate for moisture retention, Amodimethicone and Dimethicone for conditioning, and Cetearyl Alcohol, Cetyl Hydroxyethylcellulose, and Xanthan Gum for texture stability. The composition has a specific gravity of about 0.88, forming a low-density, whipped, aerated formulation for enhanced absorption. Depending on application time before rinsing, testing demonstrates 30-70% disulfide bond restoration, 60-68% split-end improvement, 40-46% complete split-end closure, 80% breakage reduction, 90% reduced wet combing force, and 24-hour frizz control in 80% humidity. The synergistic formulation strengthens, hydrates, smooths, and protects hair while maintaining lightweight, non-greasy deposition.

### TECHNICAL FIELD

The inventions disclosed herein pertain to bio-affecting and body treating compositions, specifically focusing on externally applied formulations for improving and restoring hair structure and appearance. The inventions include a rinse-off hair treatment mask containing active agents such as, for example, therapeutically effective amounts of Hydrolyzed Vegetable Protein PG-Propyl Silanetriol and Silybum Marianum Seed Extract as primary ingredients, which repair disulfide bonds to strengthen damaged hair and reduce the visual appearance of split-ends. They also include hydrating ingredients like Sodium Hyaluronate derivatives to enhance elasticity and moisture. The lightweight, low-specific-gravity composition ensures superior sensory appeal, even application, and rapid rinsing, aligning the inventions with the purpose of providing therapeutic and cosmetic benefits through external application of a bio-affecting and body treatment composition.

### DESCRIPTION OF THE RELATED ART

Hair damage is a widespread concern that affects individuals of all hair types and textures, arising from various environmental, chemical, and mechanical stressors. Daily exposure to ultraviolet radiation, pollution, fluctuating humidity, and harsh weather conditions gradually weakens the hair's natural defenses, leading to a loss of moisture, increased porosity, and an overall decline in structural integrity. These external aggressors accelerate the deterioration of the hair cuticle, the outermost protective layer, leaving the underlying cortex vulnerable to dehydration, oxidative stress, and mechanical wear. As a result, the hair becomes dry, brittle, and more susceptible to breakage, compromising its strength, elasticity, and aesthetic appeal.

The disulfide bond in human hair forms between two cysteine amino acids, bonding them together into cystine. This bond plays a crucial role in stabilizing the structure of keratin, the primary protein in hair. The sulfur atoms of two cysteine amino acids form a covalent bond, creating cystine, which is essential for the integrity of keratin protein chains. These bonds link different keratin polypeptides together, reinforcing the structure of hair and contributing to its overall durability. The number and arrangement of disulfide bonds significantly influence hair's strength, elasticity, and curliness. Unlike weaker hydrogen or ionic bonds, which can be easily broken by water or heat, disulfide bonds are among the strongest chemical bonds in hair, providing stability and resilience.

The widespread use of chemical treatments exacerbates this problem by directly altering the molecular composition of the hair fiber. Processes such as bleaching, coloring, perming, and straightening involve the use of highly reactive chemicals that break the disulfide bonds in keratin proteins. Disulfide bonds play a fundamental role in stabilizing the internal structure of hair, providing the essential cross-linking necessary for strength and resilience. When these bonds are disrupted, the keratin framework weakens, leading to a loss of elasticity, an increased tendency toward frizz, and heightened vulnerability to physical damage. Repeated chemical treatments further compound the degradation, making hair progressively weaker over time.

Mechanical stress, arising from everyday grooming practices, further contributes to hair damage. Frequent brushing, combing, and detangling, particularly when performed on wet hair, place excessive tension on the weakened fibers, leading to stretching and breakage. Heat-based styling tools, including blow dryers, curling irons, and straighteners, expose hair to high temperatures that deplete moisture and cause further breakdown of the protective cuticle. This cumulative damage results in a rough, uneven surface texture, increased susceptibility to split ends, and an overall lack of manageability, making it difficult for individuals to maintain healthy-looking hair.

A common sign of damage is the formation of split ends, which develop when the protective cuticle layer is eroded, exposing the inner cortex to further fragmentation. As the structural proteins within the fiber weaken, the strand begins to unravel, leading to visible splitting along the shaft. Split ends not only diminish the visual quality of the hair but also indicate progressive fiber breakdown, making hair more prone to snapping. Individuals seeking to maintain longer hair are particularly affected by this issue, as split ends necessitate frequent trimming, preventing the retention of length.

Another visible consequences of hair damage is the development of frizz, which arises when the cuticle layer becomes compromised and is unable to retain moisture evenly. Frizz occurs when the hair absorbs excess atmospheric moisture due to porosity imbalances, causing individual strands to swell and misalign. This issue is especially pronounced in humid environments, where the lack of an intact protective barrier allows external moisture to penetrate the cortex unchecked. Traditional anti-frizz treatments often rely on heavy silicones or waxes to coat the hair surface, temporarily masking the problem rather than addressing its root cause.

Despite the severity of these issues, many commercially available hair care solutions fail to address the fundamental causes of hair damage. Most treatments on the market focus primarily on surface-level conditioning, utilizing heavy formulations designed to provide temporary smoothness and shine. While these products may impart an immediate cosmetic improvement, they do not repair the structural deficiencies within the hair fiber. As a result, consumers find themselves trapped in a cycle of constant reapplication, experiencing diminishing returns with continued use.

The inadequacy of traditional conditioning treatments is further compounded by their lack of durability. Many formulations rely on water-soluble ingredients that rinse away easily, requiring repeated applications to maintain their effects. This transient nature fails to provide meaningful long-term benefits, leaving hair vulnerable to recurring damage. Additionally, many leave-in and deep-conditioning treatments require prolonged exposure times, making them impractical for individuals with busy lifestyles who seek effective solutions without extended waiting periods.

Another critical limitation of existing hair treatments is their inability to address multiple concerns simultaneously. Consumers frequently find themselves needing to use separate products to target frizz, breakage, hydration, and repair, resulting in complex and costly hair care routines. This fragmented approach increases the risk of product incompatibility, as layering multiple treatments can lead to excessive buildup, greasiness, or undesired heaviness, particularly for individuals with fine or low-density hair. The lack of an all-in-one solution contributes to frustration among consumers seeking comprehensive yet lightweight treatments.

Environmental considerations also play an increasingly important role in consumer preferences, with many individuals seeking hair care solutions that align with sustainability values. However, many conventional hair treatments rely on synthetic silicones, harsh surfactants, and non-biodegradable ingredients that contribute to long-term environmental damage. These formulations not only pose ecological concerns but also lead to product buildup on the scalp and hair, potentially causing irritation or contributing to further damage over time.

The need for effective treatments is particularly pronounced among individuals with severely damaged or chemically processed hair. Bleached, color-treated, or relaxed hair requires intensive restoration at the molecular level to counteract the structural breakdown caused by harsh chemical processes. Unfortunately, many current treatments do not penetrate deep enough into the hair fiber to deliver meaningful repair, instead offering superficial conditioning that fails to restore lost strength. This leaves individuals with processed hair in search of salon-based solutions, which can be expensive and time-consuming.

Time efficiency remains a major challenge in hair care, as many existing treatments require extensive application and processing times to deliver visible results. Salon-based treatments, such as bond-building procedures, can take hours and require professional application, making them impractical for regular use. Meanwhile, at-home treatments often involve multi-step routines that are cumbersome for individuals seeking fast, effective solutions. The lack of convenient yet effective options leaves many consumers without viable choices that fit their lifestyles.

Hair diversity presents another gap in the market, as many existing treatments fail to cater to the unique needs of different hair types. Individuals with textured, curly, or coily hair often struggle to find products that provide sufficient hydration without excessive weight. Because curly and coily hair types are naturally drier due to the structure of their strands, they require treatments that can penetrate deeply without causing buildup. However, many widely available products are formulated with straight or wavy hair types in mind, leaving a significant portion of consumers underserved.

Scientific validation is another area where conventional hair care solutions fall short. Many products rely on marketing claims rather than rigorous testing, leading to skepticism among consumers. Without clinical studies or quantifiable data to support their efficacy, many hair treatments fail to inspire confidence, leaving consumers to rely on anecdotal evidence or trial-and-error approaches to find effective solutions. This lack of transparency contributes to frustration and dissatisfaction with available options.

As the global hair care industry evolves, there is increasing demand for multifunctional treatments that provide both aesthetic and structural benefits. However, many formulations continue to prioritize either appearance or repair rather than integrating both aspects. Products that enhance shine and smoothness frequently neglect deeper structural repair, while those designed for bond-building often lack the lightweight conditioning benefits needed for daily use. This disconnect between market demand and product innovation has left a persistent gap in the availability of balanced, science-driven formulations.

Over the years, consumers have expressed a strong demand for treatments that provide intensive repair without the heaviness associated with traditional deep-conditioning products. However, lightweight formulations that deliver lasting improvements remain rare, forcing individuals to choose between efficacy and ease of use. The absence of such products has resulted in unmet consumer needs, leaving individuals to compromise on either long-term hair health or daily styling convenience.

Another underlying issue is the general lack of awareness surrounding the importance of molecular bond repair in achieving long-term hair strength. Many consumers remain unaware that disulfide bonds are critical to hair integrity, leading them to prioritize surface treatments that offer only temporary benefits. This knowledge gap perpetuates the cycle of repeated damage, as consumers continue using products that fail to address the root causes of hair weakness.

For individuals with severely damaged hair, the lack of effective repair options can be emotionally distressing. Many consumers report feeling helpless when faced with thinning, breakage, or excessive frizz, as conventional treatments fail to provide the restoration they need. The inability to regain healthy-looking hair affects confidence and overall satisfaction with hair care products, leading to frustration and disappointment.

The long-felt and unmet need for this invention arises from the absence of a comprehensive, scientifically validated solution that repairs damage at the molecular level, restores disulfide bonds, enhances hair texture, and delivers lasting results without heaviness. Current offerings fail to combine efficacy, convenience, and lightweight formulation in a way that meets the diverse needs of modern consumers. The invention addresses these challenges by offering a transformative approach to hair repair and care, filling a critical gap that has persisted in the market for decades.

### SUMMARY OF THE INVENTION

The inventions described in this patent application pertain, inter alia, to an advanced, rinse-off hair treatment mask composition (and method of application thereof) specifically engineered to repair and fortify structurally compromised hair while simultaneously improving appearance, manageability, smoothness, and resilience. This scientifically validated, multifunctional formulation is designed to restore disulfide bonds, significantly reduce hair breakage, improve the visual and structural integrity of split ends, enhance moisture retention, and provide long-lasting frizz control. At the core of its efficacy are two primary active ingredients: Hydrolyzed Vegetable Protein PG-Propyl Silanetriol and Silybum Marianum Seed Extract, each incorporated in therapeutically effective amounts to deliver molecular-level repair and protection. Hydrolyzed Vegetable Protein PG-Propyl Silanetriol penetrates deep into the hair shaft, reinforcing keratin structures by restoring lost disulfide bonds, thereby improving tensile strength and resistance to breakage. Silybum Marianum Seed Extract functions as a powerful antioxidant, neutralizing oxidative stress that contributes to hair fiber degradation while simultaneously protecting the restored bonds from further weakening.

Unlike conventional hair masks that rely on heavy leave-in conditioning agents, which can weigh down the hair and lead to buildup, the disclosed inventions feature a rinse-off, low-specific-gravity, aerated, whipped texture, providing a uniquely lightweight yet deeply reparative treatment. This innovative formulation leverages its reduced density to enhance deposition and absorption, allowing active ingredients to penetrate the hair fiber during a short application without leaving behind unwanted residue. The carefully controlled low specific gravity ensures that the formulation remains highly spreadable, optimizing coverage across the hair shaft while maintaining a weightless finish. The novel and non-obvious composition ensures that hydration, strength, and protection are effectively delivered without compromising natural volume or movement and are provided in a short rinse-off delivery mechanism instead of a leave-in conditioner or salon treatment

The inclusion of therapeutically effective amounts of both primary and secondary active ingredients enables targeted molecular repair, reinforcing the hair's structural integrity while simultaneously improving elasticity and moisture balance. The low-density, whipped texture further enhances rinseability, ensuring that the treatment is easily removed without diminishing its conditioning efficacy. By eliminating the drawbacks associated with traditional heavy treatments, leave-in conditioners, or labor-intensive salon procedures, this unexpectedly effective and non-obvious invention provides a groundbreaking solution for individuals seeking professional-grade hair restoration in a convenient, at-home application.

As briefly noted above, at the core of these inventions are two primary active ingredients: Hydrolyzed Vegetable Protein PG-Propyl Silanetriol and Silybum Marianum Seed Extract, both included in therapeutically effective amounts to ensure optimal performance. Hydrolyzed Vegetable Protein PG-Propyl Silanetriol is present at approximately 0.175% by weight in a sample non-limiting preferred embodiment. This functionalized protein is engineered to penetrate deeply into the hair fiber, where it actively restores broken disulfide bonds, reinforcing the keratin structure and increasing the resilience of the hair. Disulfide bonds are critical for hair strength and elasticity, but they are frequently compromised by chemical treatments, mechanical stress, and environmental exposure. By delivering targeted molecular repair, this active ingredient restores the internal integrity of the hair fiber, making it stronger, more flexible, and less prone to breakage.

Silybum Marianum Seed Extract, another key active ingredient, is included in a therapeutically effective amount of approximately 0.050% by weight in one sample, preferred, non-limiting, embodiment. This botanical extract, derived from the milk thistle plant, functions as a potent antioxidant that neutralizes free radicals and oxidative stress, which are major contributors to protein degradation in the hair. By preventing oxidative damage, this extract protects and prolongs the longevity of the repaired disulfide bonds, ensuring sustained improvements in hair strength and elasticity. Additionally, Silybum Marianum Seed Extract supports overall scalp and hair health, helping to maintain hydration, combat environmental aggressors, and promote smooth, healthy-looking hair.

To further enhance the formulation's efficacy, a combination of secondary active ingredients has been incorporated in therapeutically effective amounts to complement the primary active ingredients. Sodium Hyaluronate, included at approximately 0.0025% by weight in one sample preferred embodiment, serves as a highly effective humectant, binding moisture to the hair shaft and preventing dehydration. This ingredient is particularly beneficial for chemically treated or thermally damaged hair, as these processes strip the hair of its natural moisture. Hydroxypropyltrimonium Hyaluronate, included at approximately 0.00188% by weight of one sample preferred embodiment, provides additional conditioning benefits by forming a lightweight, hydrating film on the hair surface, reducing friction, improving detangling, and enhancing smoothness. Hydrolyzed Sodium Hyaluronate, present at approximately 0.00063% by weight in one sample preferred embodiment, features a lower molecular weight that allows it to penetrate deep into the hair cortex, delivering long-lasting hydration from within and increasing elasticity.

In addition to its hydration-enhancing properties, the formulation incorporates lightweight conditioning agents that selectively deposit on damaged areas of the hair to provide targeted repair and smoothing benefits. Dimethicone and Amodimethicone work synergistically to create a flexible, protective film on the hair fiber that locks in moisture, smooths the cuticle, and reduces frizz. These silicone-based compounds are included in specific ratios to prevent excessive buildup while ensuring that the treatment remains lightweight and effective. The amine-functionalized structure of Amodimethicone allows for selective deposition, ensuring that the most porous and damaged regions of the hair receive the greatest conditioning benefits without making the hair feel heavy or greasy.

The therapeutically effective amounts of the primary and/or secondary active ingredients in this innovative rinse-off hair treatment mask can be selected without undue experimentation based on the specific goals of the formulation, the particular selected composition, and the desired benefits. They may be varied as desired without undue experimentation to select therapeutically effective amounts for any particular desired composition. These amounts are determined through research and testing to ensure that the ingredients work synergistically to repair disulfide bonds, strengthen hair fibers, improve the visual appearance of split ends, enhance moisture retention, reduce breakage, and improve overall manageability. The precise percentage of each active ingredient may vary within an optimal range depending on the composition's overall structure, the presence of additional active or conditioning agents, and the need to balance efficacy with product stability, sensory experience, and rinseability.

For the primary active ingredients, Hydrolyzed Vegetable Protein PG-Propyl Silanetriol can be included in a therapeutically effective amount of at least about 0.175% by total weight of the composition of one sample preferred embodiment. This functionalized protein-silicone hybrid penetrates the hair cortex, restoring lost disulfide bonds and reinforcing the keratin network. However, the percentage of Hydrolyzed Vegetable Protein PG-Propyl Silanetriol may be varied within a range of approximately 0.05% to 2.00%, or more, depending on the desired intensity of bond repair and structural reinforcement. Lower amounts (around 0.05% to 0.20%) may be used in formulations designed for lighter conditioning with minimal structural modification, while higher concentrations (around 0.50% to 2.00%) (or ranges therebetween) may be included in treatments aimed at more intensive repair for chemically damaged or highly porous hair. Increasing the concentration enhances the ability of the composition to fortify hair fibers against future breakage but must be carefully balanced to avoid excessive protein buildup, which could lead to hair rigidity.

Silybum Marianum Seed Extract, another core active ingredient, is present in a therapeutically effective amount of at least about 0.050% by total weight in a sample preferred embodiment. This botanical extract delivers antioxidant protection, helping to neutralize oxidative stress and prevent environmental damage to the hair fiber. The effective range for this ingredient may vary between approximately 0.025% and 1.00%, depending on the formulation's overall goals. Lower concentrations (0.025% to 0.10%) may be sufficient for a particular composition, especially if combined with other antioxidants such as green tea extract or vitamin E, while higher concentrations (0.50% to 1.00%) (or ranges between the lower and higher potential concentrations) may be preferable in formulations designed specifically for color protection, UV defense, or anti-aging benefits. Undue experimentation can easily identify the optimum percentages for any proposed combination of ingredients since only the primary active ingredients need be varied and/or also the secondary active ingredients if desired. The inclusion of Silybum Marianum Seed Extract within these ranges (or values therebetween) ensure that the formulation maintains a balanced antioxidant profile without interfering with the absorption or performance of other key ingredients.

The secondary active ingredients, which provide hydration, conditioning, and structural support, are also included within therapeutically effective ranges that may be adjusted based on the intended function of the formulation. Sodium Hyaluronate, a highly effective humectant, is present at about 0.0025% in a preferred embodiment. However, its inclusion may range from approximately 0.001% to 0.50%, depending on the need for moisture retention and its interaction with other hydrating agents. Lower concentrations (0.001% to 0.01%) may be used in lightweight formulations aimed at normal to oily hair types, whereas higher levels (0.10% to 0.50%) may be included in ultra-hydrating masks for severely dehydrated or chemically treated hair. Again, amounts in these ranges or in values between the higher and lower levels may used as well if desired depending on the particular chosen composition and selected ingredients.

Hydroxypropyltrimonium Hyaluronate, which provides both hydration and conditioning benefits, can be included at approximately 0.00188% in one sample preferred embodiment, with a possible range of about 0.001% to 0.30%. This variation allows for optimization of detangling and cuticle-smoothing effects without overloading the formulation with cationic conditioning agents, which could lead to buildup on fine or low-porosity hair. In formulations designed for high-slip detangling and frizz control, higher amounts may be used in combination with lightweight silicones or emollients.

Hydrolyzed Sodium Hyaluronate, which features a smaller molecular weight for deeper penetration into the hair cortex, can be included at about 0.00063% in a sample preferred embodiment, with an effective range of 0.0005% to 0.20%. The lower end of this range is sufficient for formulations that already include multiple moisture-binding agents, while the higher end is suitable for treatments targeting long-lasting hydration and increased elasticity, particularly for coarse or chemically processed hair.

The ranges of these therapeutically effective amounts are determined based on the need to balance performance with product stability, absorption, and spreadability. When adjusting concentrations, the interaction between ingredients must be considered. For example, if higher levels of Hydrolyzed Vegetable Protein PG-Propyl Silanetriol are used for maximum disulfide bond repair, it may be beneficial to slightly reduce the concentration of Hydrolyzed Sodium Hyaluronate to prevent excessive stiffness in the hair fiber. Similarly, when Silybum Marianum Seed Extract is included at the higher end of its range, it may be complemented by lower amounts of other antioxidant compounds to maintain formula balance without compromising efficacy.

The formulation also considers the need for consumer-friendly application and rinseability. Higher levels of conditioning agents such as Hydroxypropyltrimonium Hyaluronate or Sodium Hyaluronate improve slip and manageability but must be kept within a controlled range to ensure that the product rinses cleanly without excessive buildup. Additionally, the aerated, low-specific-gravity nature of the formulation requires careful optimization of ingredient levels to maintain the whipped texture while allowing for efficient deposition and penetration of active compounds.

By leveraging these flexible but carefully defined therapeutic ranges, the formulation can be adjusted to meet the needs of different hair types, damage levels, and desired treatment effects. The ability to tailor ingredient concentrations within these ranges ensures that the invention remains highly versatile, capable of delivering superior hair repair, hydration, and protection while maintaining a lightweight, user-friendly application.

One of the innovative and distinguishing features of this formulation is its low-specific-gravity, whipped texture, achieved through a carefully engineered balance of water, emollients, stabilizers, aeration techniques, and structuring agents. Traditional deep-conditioning treatments tend to be heavy, dense, and difficult to rinse out, often leaving residue that can weigh down fine hair. In contrast, these inventions achieve a specific gravity of approximately 0.88, significantly lower than conventional formulations that typically range around 0.98. This is made possible through high-shear mixing techniques that incorporate fine air bubbles into the composition, along with the use of structuring agents such as Cetearyl Alcohol, Cetyl Hydroxyethylcellulose, and Xanthan Gum, which stabilize the aerated matrix. The result is a lightweight, airy texture that spreads evenly through the hair, facilitating easy application and rapid rinse-out while ensuring deep penetration of the active ingredients.

The lightweight nature of this formulation ensures that it is highly suitable for all hair types, including fine, chemically treated, and textured hair. Unlike traditional masks that may leave fine hair feeling limp or greasy, this product provides intensive hydration and repair without compromising volume. For individuals with coarser or highly porous hair, the formulation delivers long-lasting moisture retention, frizz control, and enhanced manageability. The ability to cater to diverse hair textures while maintaining effectiveness is one of the defining advantages of this innovative composition.

The scientific efficacy of this formulation has been rigorously validated through multiple performance-based studies. Disulfide bond repair testing using Confocal Raman Spectroscopy demonstrated that a single application restored approximately 20% of lost disulfide bonds, while five consecutive applications restored up to 70%. This quantifiable improvement confirms that the formulation provides deep, lasting repair at the molecular level, reinforcing the keratin network and making the hair more resistant to future damage. Wet combing force testing revealed that the treatment reduced tangling resistance by up to 90%, significantly improving ease of styling, reducing mechanical damage, and minimizing breakage caused by friction.

Another crucial benefit of the formulation is its ability to enhance the visual appearance of split ends. Split-end closure studies using stereo microscopy at 25× magnification confirmed that 68% of split ends showed measurable closure after treatment, with 46% achieving near-complete realignment. While the treatment does not permanently fuse split ends, it significantly improves their alignment, smoothing frayed fibers and making the hair appear healthier, shinier, and more polished. This benefit is particularly valuable for individuals seeking to maintain hair length without frequent trims.

Breakage resistance testing further demonstrated the protective nature of the formulation, with hair treated for ten minutes showing an 83% reduction in breakage compared to untreated hair. This remarkable improvement underscores the ability of the formulation to reinforce hair structure, making it more resilient to mechanical stressors such as brushing, combing, and heat styling. Additionally, high-humidity challenge testing confirmed that the formulation provided a 61% reduction in frizz over a 24-hour period, effectively preventing excessive moisture absorption and humidity-induced swelling of the hair cuticle.

Another unique aspect of these inventions is its enhanced rinseability, which ensures that the product does not leave behind a heavy or greasy residue. The balance of water-phase and oil-phase ingredients allows the mask to be thoroughly rinsed away while leaving behind only the beneficial conditioning agents that continue to protect and strengthen the hair. This feature makes the formulation particularly convenient for users who seek high-performance conditioning without extended washing routines.

The formulation is also aligned with sustainable and eco-conscious product development trends. Unlike traditional hair masks that rely on non-biodegradable silicones and heavy waxes, this composition incorporates plant-based emollients and biodegradable conditioning agents that minimize environmental impact. The exclusion of excessive packaging materials is facilitated by the formulation's high efficiency, as only a small amount of product is required per application to achieve effective results.

The scientifically validated performance of these inventions establishes it as a breakthrough in hair repair technology. Unlike conventional treatments that rely on anecdotal claims, this formulation has undergone rigorous laboratory testing to confirm its efficacy in repairing disulfide bonds, reducing breakage, sealing split ends, enhancing moisture retention, and providing frizz resistance. These evidence-based results provide assurance to consumers seeking a high-performance, scientifically backed hair treatment.

By combining advanced repair technology, an innovative whipped texture, and multi-functional conditioning benefits, these inventions set a new industry standard for rinse-off hair treatments. Its ability to deliver both immediate and long-term improvements in hair strength, smoothness, and hydration makes it a transformative solution in personal care. The unique combination of targeted molecular repair, effective frizz control, and effortless rinsability ensures that this formulation outperforms traditional conditioning treatments while offering a superior sensory experience. The inventions redefine the category of hair masks, providing an advanced, highly effective solution for individuals seeking comprehensive hair restoration.

The formulated composition of this innovative rinse-off hair treatment mask is designed to provide comprehensive hair repair, conditioning, and protection, but it also allows for the inclusion of additional and/or alternative ingredients to further enhance its performance and versatility. Depending on the particular selected formulation and the desired results, various deep conditioning (moisturizing) agents, strengthening components, elasticity enhancers, mood-boosting elements, stabilizers, preservatives, emollients, and pH control agents may be incorporated into the composition to customize its benefits and optimize its effectiveness for a wide range of hair types and conditions.

Deep conditioning and moisturizing agents can be selected to enhance hydration, prevent dryness, and improve the overall suppleness of the hair. These ingredients may include potent humectants such as glycerin, panthenol (provitamin B5), and aloe vera extract, which help retain moisture within the hair fiber. Additionally, plant-derived oils such as argan oil, coconut oil, avocado oil, and shea butter may be incorporated to provide lipid-based nourishment, helping to seal hydration within the hair shaft while improving its softness and smoothness. Emollients such as coconut alkanes and coco-caprylate/caprate may be included to create a lightweight, silky texture while further reducing frizz and improving the tactile feel of the hair.

For strengthening purposes, various protein-based ingredients may be utilized to reinforce the hair structure and improve resilience. In addition to Hydrolyzed Vegetable Protein PG-Propyl Silanetriol, other hydrolyzed proteins such as keratin, rice protein, silk protein, or soy protein may be added to the formulation. These proteins penetrate the hair fiber, providing structural support and filling in gaps within the damaged cuticle layer. Additionally, amino acids such as arginine, cysteine, and serine may be incorporated to enhance the composition's ability to fortify the keratin matrix, improving tensile strength and reducing breakage.

Elasticity-enhancing agents may also be included to improve the flexibility and natural movement of the hair, preventing it from becoming brittle and prone to snapping. Ingredients such as Hydroxypropyltrimonium Hyaluronate and Hydrolyzed Sodium Hyaluronate, which are already part of the formulation, serve this function by delivering multi-layer hydration and improving elasticity from within the hair cortex. Additionally, bioactive botanical extracts such as Silybum Marianum Seed Extract, green tea extract, and bamboo extract may be added to provide antioxidant protection while preserving the natural stretch and bounce of the hair fibers.

Mood-boosting elements can be incorporated to elevate the overall sensory experience of the product. Natural botanical extracts such as Banana Flower Extract, Helichrysum Italicum Extract, and Chamomile Extract may be included for their calming and soothing properties, providing a spa-like experience during use. Essential oils such as peppermint oil, lavender oil, and tea tree oil may be added not only for their pleasant fragrance but also for their scalp-stimulating and antibacterial benefits. These ingredients contribute to an enjoyable application experience while supporting overall scalp health.

To maintain the stability and longevity of the formulation, stabilizers and preservatives can be included to prevent microbial contamination and ensure consistent performance over time. The composition may include mild, non-irritating preservatives such as Sodium Benzoate, Potassium Sorbate, and Ethylhexylglycerin, which provide effective antimicrobial protection while maintaining a clean beauty profile. Stabilizing agents such as Xanthan Gum, Cetyl Hydroxyethylcellulose, and Guar Hydroxypropyltrimonium Chloride can help maintain the integrity of the formulation's texture, ensuring that the product remains uniform and does not separate over time.

Emollients are another key category of ingredients that may be adjusted or supplemented in the formulation to improve the overall feel and spreadability of the product. Lightweight emollients such as Behentrimonium Methosulfate, Cetearyl Alcohol, and Stearamidopropyl Dimethylamine contribute to the smooth application and softening effect of the composition. These ingredients create a conditioning barrier on the hair fiber, preventing moisture loss while keeping the strands soft, manageable, and frizz-free.

pH control is an essential aspect of ensuring that the formulation remains compatible with the natural pH of hair and scalp. Citric Acid or Lactic Acid may be included to adjust the pH to an optimal level, typically between 4.0 and 5.5, to maintain the integrity of the hair cuticle and prevent excessive swelling or roughness. Proper pH balance helps ensure that the hair remains smooth, shiny, and resistant to environmental stressors.

Overall, the flexibility of the formulation allows for the inclusion of various additional and/or alternative ingredients based on the specific needs of the target user group. Whether the goal is to enhance hydration, strengthen the hair, improve elasticity, boost mood, stabilize the formulation, or fine-tune pH levels, the composition can be adjusted accordingly to provide a tailored, high-performance solution. By leveraging a combination of scientifically validated active ingredients, natural extracts, and conditioning agents, this invention delivers a customizable and comprehensive approach to hair repair and enhancement, ensuring superior results for a diverse range of hair types and conditions.

The environmental profile of the inventions reflects a commitment to sustainability and consumer preferences for eco-friendly products. The use of plant-based emollients and biodegradable ingredients minimizes environmental impact, while the lightweight formulation reduces the need for excessive packaging materials. These considerations enhance the product's appeal to environmentally conscious consumers and align with broader industry trends toward sustainable innovation.

These inventions redefine the standard for rinse-off hair treatments, combining advanced science, practical usability, and environmental responsibility. Its ability to repair disulfide bonds, reduce frizz, and visually improve split ends, all within a lightweight formulation that is convenient and effective, makes it a transformative product in the field of hair care. By addressing critical gaps in existing solutions and delivering superior results, these inventions establish itself as a leader in modern hair care innovation, offering a comprehensive solution that meets the diverse needs of today's consumers.

In light of the foregoing, the following provides a simplified summary of the present disclosure to offer a basic understanding of its various parts. This summary is not exhaustive, nor does it limit the exemplary aspects of the inventions described herein. It is not designed to identify key or critical elements or steps of the disclosure, nor to define its scope. Rather, it is intended, as understood by a person of ordinary skill in the art, to introduce some concepts of the disclosure in a simplified form as a precursor to the more detailed description that follows. The specification throughout this application contains sufficient written descriptions of the inventions, including exemplary, non-exhaustive, and non-limiting methods and processes for making and using the inventions. These descriptions are presented in full, clear, concise, and exact terms to enable skilled artisans to make and use the inventions without undue experimentation, and they delineate the best mode contemplated for carrying out the inventions.

In some arrangements, a rinse-off hair treatment composition comprises a therapeutically effective amount of a Hydrolyzed Vegetable Protein-Silicone Hybrid, wherein the Hydrolyzed Vegetable Protein-Silicone Hybrid comprises Hydrolyzed Vegetable Protein PG-Propyl Silanetriol and is configured to penetrate hair fibers to restore broken disulfide bonds and reinforce keratin structures. The composition further comprises a therapeutically effective amount of a botanical antioxidant, wherein the botanical antioxidant comprises Silybum Marianum Seed Extract and is configured to neutralize oxidative stress and protect the restored disulfide bonds from further degradation. The rinse-off hair treatment composition has a specific gravity below about 0.90, forming a low-density, aerated, and easily spreadable formulation that enhances deposition and absorption while maintaining a lightweight feel. The rinse-off hair treatment composition, when applied to hair, left for at least 10 minutes, and rinsed, restores at least about 30% of lost disulfide bonds and improves the visual appearance of at least about 60% of split ends, with at least about 40% of the split ends achieving complete closure. The synergistic combination of the hydrolyzed vegetable protein-silicone hybrid and the botanical antioxidant improves the visual appearance of split ends by reinforcing the hair fiber structure at the molecular level, reducing cortical fragmentation, and enhancing cuticle alignment, resulting in smoother, healthier-looking hair.

In some arrangements, the rinse-off hair treatment composition comprises a hydrolyzed vegetable protein-silicone hybrid in an amount ranging from about 0.05% to about 2.00% by total weight of the composition, and a botanical antioxidant in an amount ranging from about 0.025% to about 1.00% by total weight of the composition.

In some arrangements, the Hydrolyzed Vegetable Protein-Silicone Hybrid is present in the rinse-off hair treatment composition in an amount of at least about 0.175% by total weight of the composition.

In some arrangements, the botanical antioxidant is present in the rinse-off hair treatment composition in an amount of at least about 0.050% by total weight of the composition.

In some arrangements, the rinse-off hair treatment composition, when applied to hair, left for at least 10 minutes, and rinsed, restores at least about 32% of lost disulfide bonds.

In some arrangements, the rinse-off hair treatment composition, when applied to hair, left for at least 10 minutes, and rinsed, improves the visual appearance of at least about 68% of split ends, with at least about 46% of the split ends achieving complete closure.

In some arrangements, the rinse-off hair treatment composition is configured to reduce hair breakage by at least about 80% after 2,000 brush strokes in repeated grooming tests.

In some arrangements, the rinse-off hair treatment composition is configured to reduce wet combing force by at least about 90% compared to untreated hair.

In some arrangements, the rinse-off hair treatment composition has a specific gravity below about 0.88.

In some arrangements, the rinse-off hair treatment composition provides a long-lasting smoothing effect by improving hair alignment and reducing frizz for at least about 24 hours after application.

In some arrangements, the rinse-off hair treatment composition is free of parabens, sulfates, and non-biodegradable silicones.

In some arrangements, the rinse-off hair treatment composition further comprises at least one secondary active ingredient selected from the group consisting of a Hyaluronic Acid Derivative, a conditioning agent, and a structuring and stabilizing agent. The Hyaluronic Acid Derivative is selected from Sodium Hyaluronate, Hydroxypropyltrimonium Hyaluronate, or Hydrolyzed Sodium Hyaluronate and is configured to enhance moisture retention and improve hair elasticity. The conditioning agent is selected from Amodimethicone, Dimethicone, or a Cationic Polymer and is configured to smooth the hair cuticle, reduce friction, and improve manageability. The structuring and stabilizing agent is selected from Cetearyl Alcohol, Cetyl Hydroxyethylcellulose, or Xanthan Gum and is configured to maintain the aerated, whipped texture of the composition and ensure uniform application. The selected secondary active ingredients work synergistically with the Hydrolyzed Vegetable Protein-Silicone Hybrid and the botanical antioxidant to improve the deposition, absorption, and performance of the rinse-off hair treatment composition while maintaining a lightweight, non-greasy feel.

In some arrangements, the at least one secondary active ingredient is present in the rinse-off hair treatment composition in the following amounts by total weight of the composition. The Hyaluronic Acid Derivative, selected from Sodium Hyaluronate, Hydroxypropyltrimonium Hyaluronate, or Hydrolyzed Sodium Hyaluronate, is present in an amount ranging from about 0.001% to about 0.50%. The cationic hyaluronic acid derivative, selected from Hydroxypropyltrimonium Hyaluronate, is present in an amount ranging from about 0.001% to about 0.30%. The Hydrolyzed Low-Molecular-Weight Hyaluronic Acid Derivative, selected from Hydrolyzed Sodium Hyaluronate, is present in an amount ranging from about 0.0005% to about 0.20%. The conditioning agent, selected from Amodimethicone, Dimethicone, or a cationic polymer, is present in an amount ranging from about 0.10% to about 5.00%. The structuring and stabilizing agent, selected from Cetearyl Alcohol, Cetyl Hydroxyethylcellulose, or Xanthan Gum, is present in an amount ranging from about 0.10% to about 4.00%. The composition retains a specific gravity below about 0.90 and maintains an aerated, whipped structure for optimal deposition and absorption into the hair fibers.

In some arrangements, a rinse-off hair treatment composition comprises a therapeutically effective amount of a Hydrolyzed Vegetable Protein-Silicone Hybrid, wherein the Hydrolyzed Vegetable Protein-Silicone Hybrid comprises Hydrolyzed Vegetable Protein PG-Propyl Silanetriol and is configured to penetrate hair fibers to restore broken disulfide bonds, reinforce keratin structures, and improve tensile strength. The composition further comprises a therapeutically effective amount of a botanical antioxidant, wherein the botanical antioxidant comprises Silybum Marianum Seed Extract and is configured to neutralize oxidative stress, protect restored disulfide bonds from degradation, and enhance overall hair resilience. The composition also includes a therapeutically effective amount of a hyaluronic acid derivative, wherein the hyaluronic acid derivative is Sodium Hyaluronate and is configured to bind moisture to the hair shaft, improve hydration retention, and enhance elasticity. Additionally, the composition comprises a therapeutically effective amount of a cationic hyaluronic acid derivative, wherein the cationic hyaluronic acid derivative is Hydroxypropyltrimonium Hyaluronate and is configured to form a lightweight hydrating film on the hair surface, reduce friction, and improve detangling and smoothness. A therapeutically effective amount of a hydrolyzed low-molecular-weight hyaluronic acid derivative, wherein the hydrolyzed low-molecular-weight hyaluronic acid derivative is Hydrolyzed Sodium Hyaluronate, is included in the composition to penetrate into the hair cortex, deliver long-lasting hydration from within, and increase flexibility.

In some arrangements, the rinse-off hair treatment composition further comprises a therapeutically effective amount of a conditioning agent, wherein the conditioning agent comprises a combination of Amodimethicone and Dimethicone and is configured to selectively deposit on damaged areas of the hair, create a flexible protective film, smooth the cuticle, and reduce frizz without excessive buildup. The composition also includes a therapeutically effective amount of a structuring and stabilizing agent, wherein the structuring and stabilizing agent comprises a combination of Cetearyl Alcohol, Cetyl Hydroxyethylcellulose, and Xanthan Gum and is configured to maintain an aerated, whipped texture, stabilize the formulation, and enhance uniform application. The composition further contains water and additional cosmetically acceptable excipients, including pH adjusters, emulsifiers, preservatives, and rheology modifiers. The rinse-off hair treatment composition has a specific gravity below about 0.90, forming a low-density, aerated, and easily spreadable formulation that enhances deposition and absorption while maintaining a lightweight, non-greasy feel.

In some arrangements, the rinse-off hair treatment composition, when applied to hair, left for at least 10 minutes, and rinsed, restores at least about 32% of lost disulfide bonds, improves the visual appearance of at least about 68% of split ends, with at least about 46% of the split ends achieving complete closure, and reduces hair breakage by at least about 80% after 2,000 brush strokes in repeated grooming tests. The synergistic combination of the Hydrolyzed Vegetable Protein-Silicone Hybrid, Botanical Antioxidant, Hyaluronic Acid Derivatives, and Conditioning Agents works to restore molecular hair integrity, reduce cortical fragmentation, enhance cuticle alignment, improve hydration retention, and provide long-lasting smoothness and frizz control, resulting in stronger, healthier, and more manageable hair.

In some arrangements, a method for restoring disulfide bonds, reducing hair breakage, and improving the visual appearance of split ends in damaged hair comprises applying a rinse-off hair treatment composition to damp hair, wherein the rinse-off hair treatment composition comprises a therapeutically effective amount of a Hydrolyzed Vegetable Protein-Silicone Hybrid, wherein the Hydrolyzed Vegetable Protein-Silicone Hybrid comprises Hydrolyzed Vegetable Protein PG-Propyl Silanetriol and is configured to penetrate hair fibers to restore broken disulfide bonds, reinforce keratin structures, and improve tensile strength. The composition further comprises a therapeutically effective amount of a botanical antioxidant, wherein the botanical antioxidant comprises Silybum Marianum Seed Extract and is configured to neutralize oxidative stress, protect restored disulfide bonds from degradation, and enhance overall hair resilience. The composition also includes a therapeutically effective amount of a Hyaluronic Acid Derivative, wherein the Hyaluronic Acid Derivative is Sodium Hyaluronate and is configured to bind moisture to the hair shaft, improve hydration retention, and enhance elasticity. Additionally, the composition comprises a therapeutically effective amount of a cationic hyaluronic acid derivative, wherein the cationic hyaluronic acid derivative is Hydroxypropyltrimonium Hyaluronate and is configured to form a lightweight hydrating film on the hair surface, reduce friction, and improve detangling and smoothness. A therapeutically effective amount of a hydrolyzed low-molecular-weight hyaluronic acid derivative, wherein the Hydrolyzed Low-Molecular-Weight Hyaluronic Acid Derivative is Hydrolyzed Sodium Hyaluronate, is included in the composition to penetrate into the hair cortex, deliver long-lasting hydration from within, and increase flexibility.

In some arrangements, the rinse-off hair treatment composition further comprises a therapeutically effective amount of a conditioning agent, wherein the conditioning agent comprises a combination of Amodimethicone and Dimethicone and is configured to selectively deposit on damaged areas of the hair, create a flexible protective film, smooth the cuticle, and reduce frizz without excessive buildup. The composition also includes a therapeutically effective amount of a structuring and stabilizing agent, wherein the structuring and stabilizing agent comprises a combination of Cetearyl Alcohol, Cetyl Hydroxyethylcellulose, and Xanthan Gum and is configured to maintain an aerated, whipped texture, stabilize the formulation, and enhance uniform application. The composition further contains water and additional cosmetically acceptable excipients, including pH adjusters, emulsifiers, preservatives, and rheology modifiers. The rinse-off hair treatment composition has a specific gravity below about 0.90, forming a low-density, aerated, and easily spreadable formulation that enhances deposition and absorption while maintaining a lightweight, non-greasy feel.

In some arrangements, the method comprises massaging the rinse-off hair treatment composition into the hair fibers for at least 30 seconds, ensuring even distribution and penetration into the hair cortex. The composition is left on the hair for at least 10 minutes to allow sufficient absorption and molecular repair, after which the composition is rinsed from the hair with water at a controlled temperature, removing excess formulation while retaining the deposited conditioning agents. The rinse-off hair treatment composition, when applied according to the method, restores at least about 30% of lost disulfide bonds, improves the visual appearance of at least about 60% of split ends, with at least about 40% of the split ends achieving complete closure, and reduces hair breakage by at least about 80% after 2,000 brush strokes in repeated grooming tests. The synergistic combination of the Hydrolyzed Vegetable Protein-Silicone Hybrid, Botanical Antioxidant, Hyaluronic Acid Derivatives, and Conditioning Agents works to restore molecular hair integrity, reduce cortical fragmentation, enhance cuticle alignment, improve hydration retention, and provide long-lasting smoothness and frizz control, resulting in stronger, healthier, and more manageable hair.

In some arrangements, the method for restoring disulfide bonds, reducing hair breakage, and improving the visual appearance of split ends in damaged hair comprises applying a rinse-off hair treatment composition to damp hair, wherein the rinse-off hair treatment composition comprises a therapeutically effective amount of a Hydrolyzed Vegetable Protein-Silicone Hybrid, a therapeutically effective amount of a Botanical Antioxidant, a therapeutically effective amount of at least one Hyaluronic Acid Derivative, a therapeutically effective amount of a Conditioning Agent, a therapeutically effective amount of a Structuring And Stabilizing Agent, water, and additional cosmetically acceptable excipients. The Hydrolyzed Vegetable Protein-Silicone Hybrid comprises Hydrolyzed Vegetable Protein PG-Propyl Silanetriol and is configured to penetrate hair fibers to restore broken disulfide bonds, reinforce keratin structures, and improve tensile strength. The botanical antioxidant comprises Silybum Marianum Seed Extract and is configured to neutralize oxidative stress, protect restored disulfide bonds from degradation, and enhance overall hair resilience. The at least one Hyaluronic Acid Derivative is selected from Sodium Hyaluronate, Hydroxypropyltrimonium Hyaluronate, and Hydrolyzed Sodium Hyaluronate, wherein each derivative contributes to moisture retention, hair elasticity, and hydration penetration into the hair cortex. The conditioning agent is selected from Amodimethicone, Dimethicone, or a cationic polymer and is configured to smooth the hair cuticle, reduce friction, and improve manageability. The structuring and stabilizing agent is selected from Cetearyl Alcohol, Cetyl Hydroxyethylcellulose, or Xanthan Gum and is configured to maintain the aerated, whipped texture of the composition and ensure uniform application. The rinse-off hair treatment composition has a specific gravity below about 0.90, forming a low-density, aerated, and easily spreadable formulation that enhances deposition and absorption while maintaining a lightweight, non-greasy feel.

In some arrangements, the rinse-off hair treatment composition comprises the Hydrolyzed Vegetable Protein-Silicone Hybrid in an amount ranging from about 0.05% to about 2.00% by total weight of the composition, the Botanical Antioxidant in an amount ranging from about 0.025% to about 1.00% by total weight of the composition, the Hyaluronic Acid Derivative in an amount ranging from about 0.001% to about 0.50% by total weight of the composition, the Cationic Hyaluronic Acid Derivative in an amount ranging from about 0.001% to about 0.30% by total weight of the composition, the Hydrolyzed Low-Molecular-Weight Hyaluronic Acid Derivative in an amount ranging from about 0.0005% to about 0.20% by total weight of the composition, the Conditioning Agent in an amount ranging from about 0.10% to about 5.00% by total weight of the composition, and the Structuring And Stabilizing Agent in an amount ranging from about 0.10% to about 4.00% by total weight of the composition. The composition retains a specific gravity below about 0.90 and maintains an aerated, whipped structure for optimal deposition and absorption into the hair fibers.

In some arrangements, the rinse-off hair treatment composition, when applied to hair, left for at least 10 minutes, and rinsed, reduces wet combing force by at least about 90% compared to untreated hair, facilitating improved detangling and hair manageability. The composition enhances lubrication along the hair fiber, reducing resistance to combing, and ensuring a smoother styling process.

In some arrangements, the rinse-off hair treatment composition, when applied according to the method, restores at least about 70% of lost disulfide bonds after five consecutive applications, demonstrating cumulative structural reinforcement of the keratin network. The formulation delivers progressive strengthening with repeated use, helping to rebuild internal hair structure and minimize signs of damage over time.

In some arrangements, the improvement in the visual appearance of split ends, including at least about 40% of split ends achieving complete closure, is maintained for at least 72 hours post-treatment, resulting in long-lasting cuticle realignment and smoother hair texture. The treatment provides prolonged protection against further splitting and fraying, leading to visibly healthier and more manageable hair.

In some arrangements, the rinse-off hair treatment composition reduces frizz and flyaways for at least 24 hours after treatment and maintains hair alignment even under exposure to 80% relative humidity, demonstrating superior moisture resistance and cuticle smoothing effects. The formulation creates a humidity-resistant barrier on the hair surface, preventing excessive water absorption that leads to frizz and uncontrolled texture expansion. The synergistic effect of the active ingredients ensures long-lasting shine, smoothness, and manageability, making the hair more resistant to environmental fluctuations.

The following description and claims, in conjunction with the drawings—all integral parts of this specification—will clarify various features and characteristics of the current technology. Like reference numerals in the figures correspond to similar parts, enhancing understanding of the technology's methods of operation and the functions of related structural elements, as well as the synergies and economies of their combinations. Some of the processes or procedures described here may be implemented, in whole or in part, as computer-executable instructions recorded on computer-readable media, configured as computer modules, or in other computer constructs. These steps and functionalities may be executed on a single device or distributed across multiple devices interconnected with one another. However, it is important to acknowledge that the drawings primarily serve for descriptive and illustrative purposes and are not intended to delineate the limits of the invention. Unless contextually evident, the singular forms of “a,” “an,” and “the” used throughout the specification and claims should be interpreted to include their plural counterparts.

### BRIEF DESCRIPTION OF DRAWINGS

FIG. 1A illustrates a split-end closure scoring scale, depicting hair fibers rated at levels 1 (worst performance) and 2 based on their degree of closure before and after treatment. The scale ranges from no closure to minimal partial closure, with microscopic imaging used to assess and quantify changes.

FIG. 1B expands on the split-end closure scoring scale, showing hair fibers rated at levels 3 and 4, representing a moderate to reasonable degree of closure before and after treatment. The scale confirms the visual improvement of split ends following the application of the hair mask.

FIG. 1C presents the highest level of the split-end closure scale, showing a hair fiber rated at level 5 (best performance), indicating complete closure after treatment. This figure demonstrates the product's ability to visually enhance split ends to the greatest extent possible.

FIG. 2 provides before-and-after microscopic images of Test Hair Fiber No. 1, demonstrating the treatment's impact on split-end closure. The images visually validate the product's ability to improve hair fiber alignment and reduce visible split ends.

FIG. 3 presents before-and-after images of Test Hair Fiber No. 2, further supporting the effectiveness of the treatment in achieving a higher degree of closure. The comparison illustrates significant improvements in fiber cohesion after application.

FIG. 4 shows before-and-after images of Test Hair Fiber No. 3, depicting the reduction of split ends following treatment. The images confirm that the formulation improves the appearance of hair fibers by sealing and smoothing the damaged ends.

FIG. 5 illustrates the before-and-after images on Test Hair Fiber No. 4, showcasing additional evidence of enhanced split-end appearance. The treated fiber exhibits greater uniformity and alignment compared to its untreated counterpart.

FIG. 6 provides another set of before-and-after images for Test Hair Fiber No. 5, reinforcing the cumulative effectiveness of multiple applications. The treatment results in a more polished and refined hair fiber structure.

FIG. 7 illustrates the structure of an undamaged disulfide bond within a healthy hair fiber, highlighting the covalent bond between sulfur atoms on different keratin polypeptide chains. This figure establishes a reference for understanding the structural integrity of healthy hair.

FIG. 8 depicts a broken disulfide bond in a damaged hair fiber, showing the separation of sulfur atoms within keratin polypeptide chains. This visualization emphasizes the structural weakening that occurs due to chemical, mechanical, or environmental stress.

FIG. 9 illustrates the application of the hair treatment mask to a damaged hair fiber, showing the interaction of the composition with the broken disulfide bonds. This figure conveys the targeted action of the formulation in restoring hair integrity.

FIG. 10 presents a repaired disulfide bond aftertreatment with the hair mask, showing the restored covalent bond between sulfur atoms on keratin polypeptide chains. This figure confirms the product's efficacy in repairing hair at the molecular level, validating its disulfide bond restoration capabilities.

### DETAILED DESCRIPTION

The inventions include various rinse-off hair treatment compositions meticulously designed to repair, strengthen, and enhance damaged hair at both a molecular and surface level. Unlike traditional hair masks that rely primarily on surface conditioning agents, this formulation actively restores the structural integrity of the hair by repairing broken disulfide bonds, reducing breakage, sealing split ends, and improving overall smoothness and manageability. The composition is uniquely characterized by its low specific gravity, which results in a lightweight, whipped texture that spreads evenly throughout the hair, allowing for thorough application and easy rinsing. This innovative formulation delivers both immediate and long-term benefits by reinforcing the keratin structure, providing hydration, and protecting the hair from further environmental and mechanical stressors.

Central to the effectiveness of this composition are two primary active ingredients, Hydrolyzed Vegetable Protein PG-Propyl Silanetriol and Silybum Marianum Seed Extract. Hydrolyzed Vegetable Protein PG-Propyl Silanetriol is a functionalized protein that penetrates deep into the hair fiber to restore broken disulfide bonds, which are crucial for maintaining hair strength, flexibility, and elasticity. This ingredient actively adheres to weakened areas within the hair's protein matrix, reinforcing structural integrity and making the hair more resistant to future damage from chemical treatments, heat styling, and environmental exposure. Silybum Marianum Seed Extract serves as a powerful antioxidant that helps neutralize free radicals and oxidative damage, which are major contributors to hair degradation. By preventing oxidative stress, this ingredient enhances the longevity of hair repair, ensuring that the hair remains strong and protected over time.

The composition is further enhanced by a series of secondary active ingredients that work in concert to support hydration, cuticle repair, and fiber smoothness. Sodium Hyaluronate, Hydroxypropyltrimonium Hyaluronate, and Hydrolyzed Sodium Hyaluronate function as highly effective humectants that deliver multi-layer hydration by binding moisture within the hair shaft. These Hyaluronic Acid Derivatives, each with varying molecular weights, ensure that hydration is delivered at different depths within the hair structure, from the cuticle layer to the deeper cortex. By restoring internal moisture levels, these ingredients improve elasticity, prevent brittleness, and enhance the overall manageability of the hair. The combined hydration effects reduce the risk of breakage, enhance flexibility, and promote a healthier hair fiber that is more resistant to external stressors.

The inventions utilize a specific gravity that is carefully controlled to optimize the deposition and absorption of conditioning agents onto hair fibers. This specific gravity (@0.88) is achieved through the precise formulation of the treatment, incorporating a balance of water, conditioning agents, and film-forming polymers that create a lightweight yet effective coating. By adjusting the density of the formulation, the treatment ensures even distribution and penetration into the hair cortex without excessive buildup. This controlled specific gravity is particularly beneficial as it enhances the product's ability to restore damaged disulfide bonds, reduce breakage, and improve manageability while maintaining a natural feel. The result is a treatment that delivers intensive repair and conditioning benefits without weighing the hair down, making it suitable for all hair types, including chemically treated and fine hair.

In addition to hydration, the formulation includes advanced conditioning agents such as Dimethicone and Amodimethicone, which selectively deposit on the most damaged areas of the hair to create a lightweight, protective barrier. These silicone-based compounds provide long-lasting smoothness, control frizz, and enhance shine without causing buildup or heaviness. The amine-functionalized properties of Amodimethicone enable targeted conditioning, ensuring that the most porous and compromised regions of the hair receive the greatest benefit. This selective deposition technology ensures that hair remains soft, smooth, and well-protected while maintaining its natural volume and movement.

A key differentiator of these inventions are their low specific gravity, which imparts a unique lightweight, airy, and whipped texture to the product. Unlike conventional deep-conditioning treatments that tend to be heavy and greasy, this formulation achieves a low-density structure that allows for easy application and rinsing. The composition has a specific gravity of approximately 0.88, compared to traditional hair masks that typically range around 0.98. This is accomplished through a combination of structuring agents, emulsifiers, and aeration techniques. Cetearyl Alcohol, a fatty alcohol that stabilizes emulsions, contributes to the whipped texture by trapping air within the formulation and maintaining its light, spreadable consistency. Behentrimonium Methosulfate further supports the formulation's stability while providing excellent detangling benefits. The inclusion of Cetyl Hydroxyethylcellulose and Xanthan Gum ensures that the aerated structure remains intact over time, preventing phase separation and maintaining a uniform texture.

Another significant advantage of this composition is its ability to restore disulfide bonds, which are critical for maintaining the strength and elasticity of the hair. Laboratory testing using Confocal Raman Spectroscopy has demonstrated that a single application of this treatment restores approximately 20 percent of lost disulfide bonds, while five consecutive treatments restore up to 70 percent. This quantifiable improvement confirms that the formulation is capable of reinforcing the internal keratin network, making the hair more resilient to future damage. Unlike surface-level conditioning agents that offer temporary benefits, this technology actively repairs and strengthens the hair at a molecular level, ensuring long-lasting results.

The effectiveness of this formulation in improving the visual appearance of split ends has also been validated through advanced microscopic imaging techniques. Split-end closure testing using stereo microscopy at 25× magnification revealed that 68 percent of split ends exhibited visible closure after treatment, with 46 percent achieving near-complete closure. The product does not permanently fuse split ends but significantly improves their alignment, making the hair appear healthier, more polished, and free of frayed or jagged edges. The ability to visually enhance split ends is particularly valuable for individuals who want to maintain hair length without the need for frequent trimming.

Breakage resistance is another core benefit of this formulation. Mechanical stress, such as daily brushing, combing, and heat styling, can weaken the hair and cause strand fragmentation. To assess the composition's ability to prevent breakage, anti-breakage testing was conducted using automated repeated grooming simulations. The results showed that the treatment reduced breakage by 83 percent after a 10-minute application and by 77 percent after a three-minute application. This significant improvement demonstrates that the formulation effectively reinforces the hair fiber, reducing vulnerability to mechanical damage and ensuring stronger, more resilient strands.

The composition also improves hair detangling and manageability, making it easier to comb through hair with reduced mechanical stress. Wet combing tests performed using an Instron tensile tester revealed that the formulation reduced combing force by at least 90 percent after at least a three-minute application. This means that hair treated with the product experiences significantly less friction and tangling, minimizing damage during everyday grooming and styling.

Another standout feature of the inventions is its ability to combat frizz, particularly in humid conditions. High-humidity challenge tests confirmed that the formulation reduced frizz by 61 percent over a 24-hour period, providing long-lasting humidity resistance. Frizz occurs when external moisture disrupts the hair's cuticle layer, causing uneven swelling and an unmanageable appearance. The formulation's ability to create a lightweight barrier helps lock in internal hydration while preventing external humidity from affecting the hair fiber. This ensures that hair remains smooth, sleek, and frizz-free even in challenging environmental conditions.

The formulation is designed to be compatible with a diverse range of hair types, including fine, thick, curly, and textured hair. Fine hair benefits from the lightweight nature of the formula, which delivers hydration and repair without weighing the hair down. Textured and curly hair, which is prone to dryness and frizz, experiences enhanced moisture retention, improved elasticity, and greater definition. This inclusivity ensures that the formulation provides universal benefits, catering to individuals with varying hair concerns.

Sustainability is also a key consideration in the development of this composition. The formulation incorporates plant-based and biodegradable ingredients wherever possible, reducing its environmental impact. The low-density texture also contributes to improved efficiency, as less product is required per application to achieve effective results. The exclusion of non-biodegradable silicones and the use of mild, non-irritating preservatives such as Sodium Benzoate and Potassium Sorbate enhance its appeal as a clean beauty formulation.

The scientific validation of this formulation establishes it as a credible and high-performance hair care solution. Unlike conventional treatments that rely on anecdotal claims, these inventions have undergone rigorous laboratory testing to confirm its efficacy in disulfide bond repair, split-end smoothing, breakage prevention, frizz reduction, hydration enhancement, and detangling improvement. These evidence-based results provide assurance to consumers seeking a scientifically backed treatment that delivers measurable improvements in hair health.

By combining advanced repair technology, a lightweight whipped texture, and multi-functional benefits, these inventions set a new standard for rinse-off hair treatments. Its ability to deliver both immediate and long-term improvements in hair strength, smoothness, and hydration makes it a breakthrough innovation in the personal care industry. The unique combination of targeted molecular repair, effective frizz control, and effortless rinsability ensures that this formulation outperforms traditional conditioning treatments while offering a superior sensory experience. These inventions redefine the category of rinse-off hair treatment masks, providing an advanced and effective solution for individuals seeking high-performance hair restoration.

Sample deep conditioner actives in the inventive formulation can be a curated blend of one or more of moisturizing, strengthening, elasticity-enhancing, and/or mood-boosting ingredients designed to improve hair structure, hydration, and sensory experience. Each active serves a specific function in enhancing the performance of the conditioner, ensuring that hair is deeply nourished, repaired, and protected.

The moisturizing (deep conditioning) actives can replenish hydration and preventing dryness. Vita Hya-Hair Repair is a blend containing Sodium Hyaluronate, Hydrolyzed Sodium Hyaluronate, and Hydroxypropyltrimonium Hyaluronate, which work together to provide multi-layer hydration by binding moisture at different levels of the hair shaft. These ingredients ensure that hair retains water for an extended period, improving elasticity and reducing brittleness. The inclusion of Glycerin, a humectant, further enhances moisture retention by drawing water into the hair fiber. Actimilk, which consists of Argan Water, Argania Spinosa Kernel Oil, Glycerin, and Lysolecithin, can provide both hydration and nourishment, with argan oil contributing essential fatty acids that protect and smooth the hair. Crodarom Banana Flower EC, derived from Musa Sapientum (Banana) Flower Extract, could add a natural conditioning effect, improving the overall softness and manageability of hair. PhytoVie Defense, a Brassica Campestris/Aleurites Fordi Oil Copolymer, could act as a natural film-forming agent, providing a lightweight protective shield against environmental stressors, such as pollution and humidity.

The strengthening actives in the formulation can help reinforce the hair structure, making it more resistant to breakage and mechanical stress. Keravis NSP ET, which contains Hydrolyzed Vegetable Protein PG-Propyl Silanetriol, can fortify the hair fiber by penetrating the cortex and rebuilding weakened keratin structures. This ingredient improves both the tensile strength and flexibility of the hair, making it less prone to breakage. Actimilk, already mentioned for its moisturizing benefits, could also contributes to hair strengthening by providing lipid nourishment and reinforcing the cuticle. PhytoVie Defense, in addition to its protective properties, enhances the hair's resistance to daily wear and tear, particularly against environmental aggressors that contribute to oxidative damage.

For improving hair elasticity, the formulation could include Keravis NSP ET, which works to increase the flexibility of hair strands by binding to keratin proteins. Kerasylium, a combination of Maltodextrin and Silybum Marianum Seed Extract, supports elasticity by delivering antioxidant benefits while improving hair's natural stretch and bounce. VitaSoft Peel, which contains Succinic Acid, gently enhances hair elasticity by supporting the restructuring of keratin fibers. These ingredients collectively help prevent hair from becoming rigid and brittle, ensuring that it retains its natural movement and ability to withstand stretching forces.

Mood-boosting actives can contribute to the overall sensory experience of using the deep conditioner. Crodarom Banana Flower EC, aside from its conditioning benefits, provides a pleasant natural fragrance and soothing effect. CORTINHIB G, which contains Helichrysum Italicum Extract, is known for its calming and anti-inflammatory properties, making it beneficial for scalp health and overall user experience. TiMOOD, a blend of Zanthoxylum Alatum Fruit Extract, Glycerin, and Pentylene Glycol, is included for its ability to promote relaxation and a positive sensory response. Lecigel, which combines Sodium Acrylates Copolymer and Lecithin, contributes to the luxurious, smooth feel of the formulation, improving its spreadability.

Various of the above deep conditioning actives can work together to create a formulation that moisturizes, strengthens, improves elasticity, and enhances the overall experience of use. The blend of humectants, proteins, botanical extracts, and protective agents ensures that the conditioner delivers deep nourishment, structural reinforcement, and sensory appeal, making it a highly effective solution for repairing and revitalizing hair.

One sample embodiment of the present inventions included the following INCI ingredients and corresponding percentages.

TABLE 1
Exemplary Formulation for Sample Preferred Embodiment

| INCI Ingredient | Percentage by Weight |
|---|---|
| Water | 74.78143% |
| Cetearyl Alcohol | 8.50000% |
| Dimethicone | 3.00000% |
| Propanediol | 2.00000% |
| Coconut Alkanes | 1.88000% |
| Amodimethicone | 1.20000% |
| Dipropylene Glycol | 1.05000% |
| Behentrimonium Methosulfate | 1.00000% |
| Cocos Nucifera (Coconut) Oil | 1.00000% |
| Stearamidopropyl Dimethylamine | 0.75000% |
| Glycerin | 0.53630% |
| Cetrimonium Chloride | 0.51500% |
| Cetyl Hydroxyethylcellulose | 0.50000% |
| Sodium Levulinate | 0.47600% |
| Polysilicone-29 | 0.45000% |
| Citric Acid | 0.34190% |
| Guar Hydroxypropyltrimonium Chloride | 0.30000% |
| Sodium Benzoate | 0.26600% |
| Trideceth-12 | 0.24000% |
| Hydrolyzed Vegetable Protein PG-Propyl | 0.17500% |
| Silanetriol | |
| Mentha Piperita (Peppermint) Oil | 0.15870% |
| Maltodextrin | 0.15000% |
| Coco-Caprylate/Caprate | 0.12000% |
| Menthol | 0.12000% |
| Argania Spinosa Kernel Oil | 0.10000% |
| Melaleuca Alternifolia (Tea Tree) Leaf Oil | 0.08037% |
| Melia Azadirachta Leaf Extract | 0.07000% |
| Arginine | 0.05000% |
| Silybum Marianum Seed Extract | 0.05000% |
| Musa Sapientum (Banana) Flower Extract | 0.03000% |
| Melia Azadirachta Flower Extract | 0.02000% |
| Potassium Sorbate | 0.01500% |
| Beta-Caryophyllene | 0.01050% |
| Lysolecithin | 0.01000% |
| Xanthan Gum | 0.01000% |
| 1,2-Hexanediol | 0.00750% |
| Sodium Hyaluronate | 0.00250% |
| Hydroxypropyltrimonium Hyaluronate | 0.00188% |
| Ethylhexylglycerin | 0.00063% |
| Hydrolyzed Sodium Hyaluronate | 0.00063% |
| Helichrysum Italicum Extract | 0.00025% |

Preferably, the inventive composition will include at least about 0.050% Silybum Marianum Seed Extract and at least about 0.175% Hydrolyzed Vegetable Protein PG-Propyl Silanetriol. These amounts were sufficient to achieve the test results described herein. However, higher percentages may be utilized if desired and may provide even further disulfide bond repair and improved visual appearance of split ends. In some embodiments, it is also preferable to include one or more of at least about 0.00250% Sodium Hyaluronate, about 0.00188% Hydroxypropyltrimonium Hyaluronate, and about 0.00063% Hydrolyzed Sodium Hyaluronate. Again, higher percentages may be incorporated if desired.

As noted above, various tests were performed to evaluate the efficacy of the inventive composition. These tests were designed to measure the formulation's ability to repair hair damage, strengthen the hair fiber, improve manageability, reduce breakage, enhance moisture retention, and control frizz. The tests performed include:
a. Split-End Closure Evaluation via Stereo Microscopy (25× Magnification): This test assessed the visual improvement of split ends before and after treatment. 68% of split ends exhibited measurable closure, and 46% achieved near-complete closure after application. While the formulation does not permanently fuse split ends, it significantly smooths and realigns fibers, making hair appear healthier.
b. Disulfide Bond Repair Testing via Confocal Raman Spectroscopy: This test measured the extent of disulfide bond restoration after chemical damage such as bleaching. The results showed that a single application restored approximately 20% of lost disulfide bonds, and five consecutive applications restored up to 70%. This confirmed the formulation's ability to reinforce the internal keratin structure at a molecular level.
c. Frizz Control and Humidity Resistance Testing via High-Humidity Challenge: Hair samples were exposed to 80% relative humidity for 24 hours, and frizz levels were analyzed using image software. The formulation reduced frizz by 61% over 24 hours, proving its ability to create a lightweight, humidity-resistant barrier.
d. Anti-Breakage Testing Using Automated Repeated Grooming Simulation: A mechanical stress test simulated 2,000 strokes of brushing and combing to assess hair strength. The results demonstrated that the formulation reduced breakage by 83% after a 10-minute application and by 77% after a three-minute application, confirming its ability to reinforce hair structure against mechanical damage.
e. Wet Combing Force Testing Using an Instron Tensile Tester: This test measured the force required to comb through wet hair treated with the formulation. The results indicated a 90% reduction in combing force after 10 minutes and 94% after three minutes, confirming significant improvements in detangling and reducing friction-related damage.

As explained herein, these tests comprehensively validate the effectiveness of the formulation in repairing disulfide bonds, reducing breakage, sealing split ends, improving manageability, enhancing moisture retention, and providing frizz resistance.

Regarding split-end closure, a controlled study to evaluate the effectiveness of the inventive rinse-off composition in improving the appearance of split ends in chemically treated and mechanically stressed hair. The goal of the study was to quantify the degree of split-end closure after applying the treatment and compare the results to the baseline condition. The experiment followed a standardized methodology, ensuring accurate tracking of individual hair fibers before and after treatment.

The study aimed to determine the ability of the conditioning mask to realign, seal, and smooth split-end fibers in hair that had been subjected to bleaching and repeated brushing. Hair samples consisted of European Medium Brown hair tresses, which were first chemically processed (bleached) and mechanically damaged through repeated brushing cycles to induce split ends.

To ensure precision and consistency, fifty (50) individual split-end fibers were randomly selected from each test cell. Each fiber was tagged and labeled so that it could be tracked throughout the experiment. These fibers served as a representative sample of hair strands with visible split ends to evaluate the impact of the treatment.

Regarding application process and treatment standardization, the following steps were followed to ensure consistency in treatment application: (1) Fifty fibers with visible split ends were randomly selected from each test cell and individually labeled. Each fiber was assigned a unique identifier to allow precise tracking before and after treatment. (2) Each of the 50 split-end fibers was imaged before treatment using a stereo microscope at 25× magnification. These high-resolution baseline images documented the size, shape, and severity of the split ends before any product was applied. (3) The labeled and imaged fibers were reintroduced into the associated hair tress from which they were originally selected. This ensured that the treated fibers remained in their natural environment within the full hair tress. (4) Each tress was treated with the inventive composition according to a standardized procedure. First, the mask was applied at 20% of the tress weight (0.20 ml per gram of hair) to ensure an even and measurable application.

Next, the treatment was massaged into the tress for 30 seconds to ensure uniform distribution and product penetration. Third, the product was left on the tress for 10 minutes, allowing the active ingredients to interact with the damaged hair structure. Fourth, the hair was rinsed for 30 seconds using an Intelifaucet set to 40° C. (104° F.) at a controlled flow rate of 1 gallon per minute (GPM) to mimic real-world washing conditions. (5) After treatment, the tresses were left to air dry overnight in a controlled environment to allow the product to fully absorb and set into the hair structure. (6) Once dried, the same 50 fibers that were originally imaged at baseline were removed again and reimaged using the same stereo microscopy technique. The post-treatment images were compared to the baseline images to assess the degree of closure and realignment of the split ends.

To objectively measure the effectiveness of the treatment, researchers assigned each fiber a numerical rating from 1 to 5 based on the degree of closure observed in the post-treatment images. The grading scale used was as follows: Grade 1: No closure (split end remains fully open with no visible improvement); Grade 2: Minimal closure (less than 30% of the split end is closed); Grade 3: Moderate partial closure (30-70% of the split end is closed); Grade 4: Significant closure (greater than 70% closure, but not fully sealed); and Grade 5: Complete closure (split end is fully closed and visually undetectable). The numerical ratings assigned to each fiber provided a quantitative measure of the treatment's effectiveness, ensuring that results were scientifically valid and reproducible.

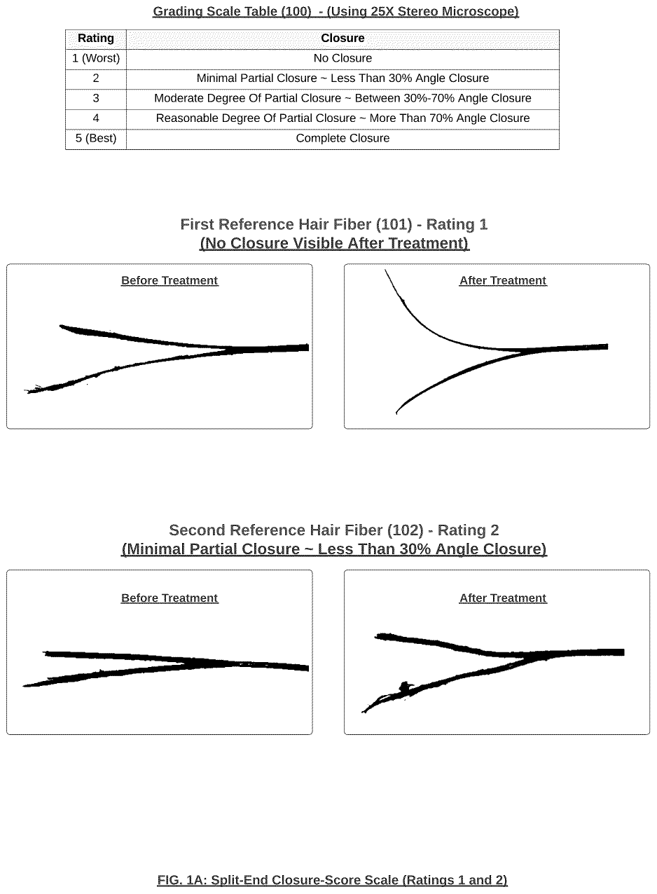

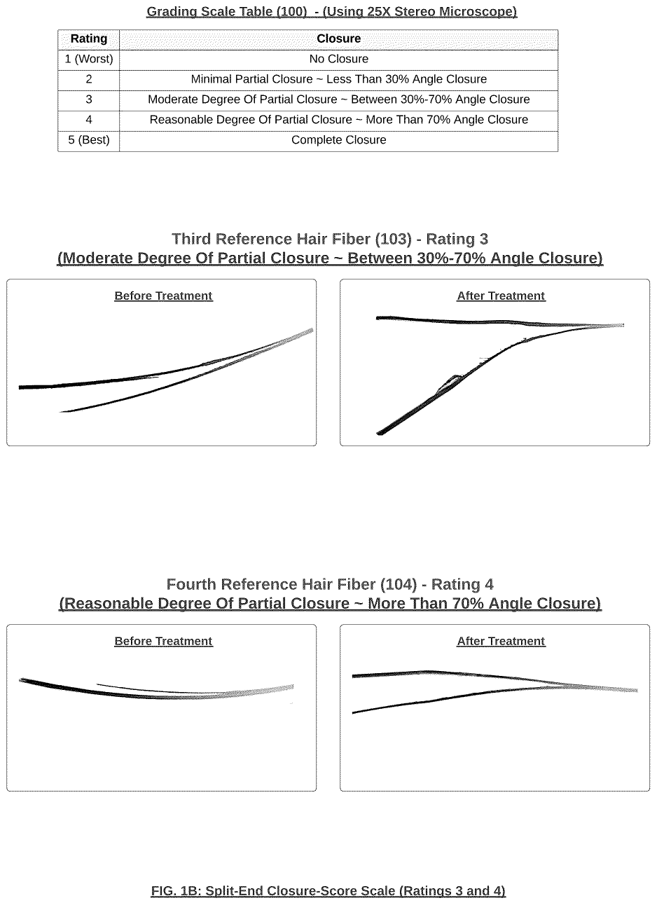

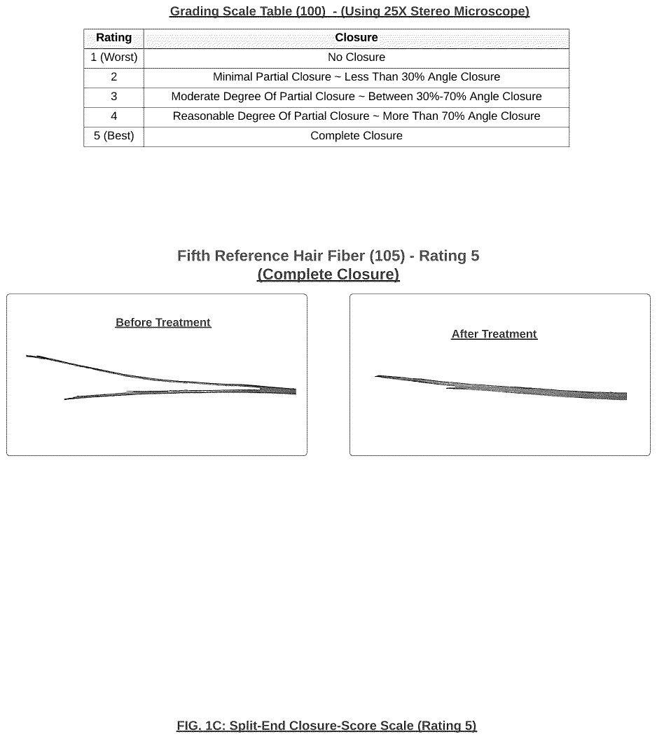

For reference, FIGS. 1A, 1B, and 1C collectively illustrate the split-end closure scoring scale used to assess and categorize the degree of closure of split ends before and after treatment. These figures establish a standardized grading system for evaluating the effectiveness of a hair treatment composition in improving the visual appearance of split ends. The scale, which ranges from 1 to 5, quantifies the extent of closure in terms of percentage of angle reduction, providing both descriptive criteria and microscopic imagery to support the evaluation process. Each figure contains a “Grading Scale Table (100),” which was created using a 25× stereo microscope and details the classification of hair fibers based on their observed closure levels. The inclusion of labeled hair fiber images at various rating levels provides visual confirmation of the classification system, allowing for consistent and reproducible assessments of split-end improvement.

An analysis of the before-and-after images demonstrate the effectiveness of the Deep Conditioning Hair Mask in significantly improving the appearance of split ends. 68% of the tested split-end fibers showed measurable closure (Grade 3 and above), indicating that the treatment realigned and sealed the split fibers to some extent in the majority of tested strands. 46% of the tested fibers achieved complete closure (Grade 5), meaning that nearly half of the split-end fibers were fully sealed after the single treatment. 22% of the fibers achieved partial closure (Grades 3 and 4), showing noticeable but incomplete repair. The overall average closure rating across all fibers was 3.66 out of 5, confirming that most fibers demonstrated a moderate to significant improvement in split-end appearance. Some fibers (32%) showed little to no closure (Grades 1 and 2), indicating that while the treatment was effective for the majority, certain hair strands were either too severely damaged or required repeated treatments for optimal repair.

For more detailed reference, a complete scoring of all 50 fibers is set forth below.

TABLE 2
Split-End Closure Grades for Each Tested Fiber

| Hair Fiber # | Closure Rating |
|---|---|
| 1 | 5 |
| 2 | 5 |
| 3 | 3 |
| 4 | 5 |
| 5 | 5 |
| 6 | 3 |
| 7 | 2 |
| 8 | 5 |
| 9 | 5 |
| 10 | 5 |
| 11 | 2 |
| 12 | 2 |
| 13 | 5 |
| 14 | 3 |
| 15 | 5 |
| 16 | 5 |
| 17 | 5 |
| 18 | 3 |
| 19 | 2 |
| 20 | 2 |
| 21 | 2 |
| 22 | 3 |
| 23 | 3 |
| 24 | 4 |
| 25 | 2 |
| 26 | 5 |
| 27 | 3 |
| 28 | 5 |
| 29 | 5 |
| 30 | 2 |
| 31 | 5 |
| 32 | 3 |
| 33 | 2 |
| 34 | 2 |
| 35 | 2 |
| 36 | 5 |
| 37 | 4 |
| 38 | 2 |
| 39 | 2 |
| 40 | 2 |
| 41 | 2 |
| 42 | 5 |
| 43 | 4 |
| 44 | 5 |
| 45 | 5 |
| 46 | 5 |
| 47 | 5 |
| 48 | 2 |
| 49 | 5 |
| 50 | 5 |

This study confirmed that the inventive rinse-off hair treatment mask provides substantial improvement in the appearance of split ends, effectively smoothing frayed hair fibers and reducing the visibility of damage. While the formulation does not permanently fuse split ends, it significantly reduces the perception of hair damage by sealing and aligning the cuticle layers, making the hair appear healthier and more polished.

This study demonstrates that even a single brief application of the rinse-off treatment mask provided quantifiable improvements in split-end appearance, making it an effective solution for individuals seeking to restore smoothness and manageability to damaged hair. The results suggest that multiple applications may provide cumulative benefits, further improving fiber alignment and preventing split-end progression over time.

For reference, FIGS. 1A, 1B, and 1C collectively illustrate the split-end closure scoring scale that was used to assess and categorize the degree of closure of split ends before and after treatment. These figures establish a standardized grading system for evaluating the effectiveness of a hair treatment composition in improving the visual appearance of split ends. The scale, which ranges from 1 to 5, quantifies the extent of closure in terms of percentage of angle reduction, providing both descriptive criteria and microscopic imagery to support the evaluation process. Each figure contains a “Grading Scale Table (100),” which was created using a 25× stereo microscope and details the classification of hair fibers based on their observed closure levels. The inclusion of labeled hair fiber images at various rating levels provided visual confirmation of the classification system, allowing for consistent and reproducible assessments of split-end improvement.

FIG. 1A introduces the first two levels of the grading scale, titled “Split-End Closure-Score Scale (Ratings 1 and 2).” This figure defines the lowest two ratings, representing hair fibers with minimal or no closure. A rating of 1 is labeled “No Closure,” indicating that the split ends remain entirely open, showing no reduction in the separation between fiber strands. A microscopic image of a “First Reference Hair Fiber (101)” rated at level 1 is included, showing a fully open and untreated split end. A rating of 2 is described as “Minimal Partial Closure~Less Than 30% Angle Closure,” indicating that the hair fibers have slightly realigned but still exhibit a visible gap greater than 70% of the original split. The corresponding image of a “Second Reference Hair Fiber (102)” rated at level 2 shows a slight improvement in alignment, but the damage remains evident.

FIG. 1B expands upon the scale by presenting “Split-End Closure-Score Scale (Ratings 3 and 4).” This figure introduces mid-level closure categories that reflect more substantial improvements in the appearance of split ends. A rating of 3 is labeled “Moderate Degree of Partial Closure~Between 30%-70% Angle Closure,” signifying that the split ends have partially realigned, and the separation between the hair fiber strands has been reduced to between 30% and 70% of its original angle. The microscopic image of a “Third Reference Hair Fiber (103)” rated at level 3 shows an observable reduction in the split-end angle, with fibers exhibiting significant partial closure. A rating of 4 is described as “Reasonable Degree of Partial Closure~More Than 70% Angle Closure,” meaning that the majority of the split-end gap has been closed, with only minor separation still visible. The microscopic image of a “Fourth Reference Hair Fiber (104)” rated at level 4 confirms this high degree of closure, as the fibers appear much more aligned and restructured, though some residual opening remains.

FIG. 1C completes the scale by displaying the highest level of split-end closure, titled “Split-End Closure-Score Scale (Rating 5).” This figure represents the ideal outcome of split-end treatment, showing a complete visual improvement of the fiber structure. A rating of 5 is labeled “Complete Closure,” indicating that the split ends have fully realigned, with no visible separation remaining between the fibers. The microscopic image of a “Fifth Reference Hair Fiber (105)” rated at level 5 provides clear visual evidence of full closure, demonstrating that the split fibers have merged into a singular, cohesive structure. This level of closure is the maximum achievable result and represents the highest standard of treatment efficacy.

Together, FIGS. 1A, 1B, and 1C established a comprehensive, scientifically validated system for assessing the visual improvement of split ends. The structured grading scale enabled precise measurement of treatment effectiveness, offering a quantifiable method to evaluate how well a formulation improves the appearance of damaged hair fibers. The inclusion of microscopic images provides essential reference points, ensuring that evaluations remain consistent across different applications and studies. By categorizing closure into five distinct levels, these reference figures helped to assess incremental improvements, helping to determine the relative efficacy of hair treatment compositions. Through the detailed classification of split-end closure levels and the visual support provided by microscopic imagery, FIGS. 1A, 1B, and 1C collectively provided an objective and reproducible framework for measuring and documenting improvements in hair fiber integrity provided by this invention.

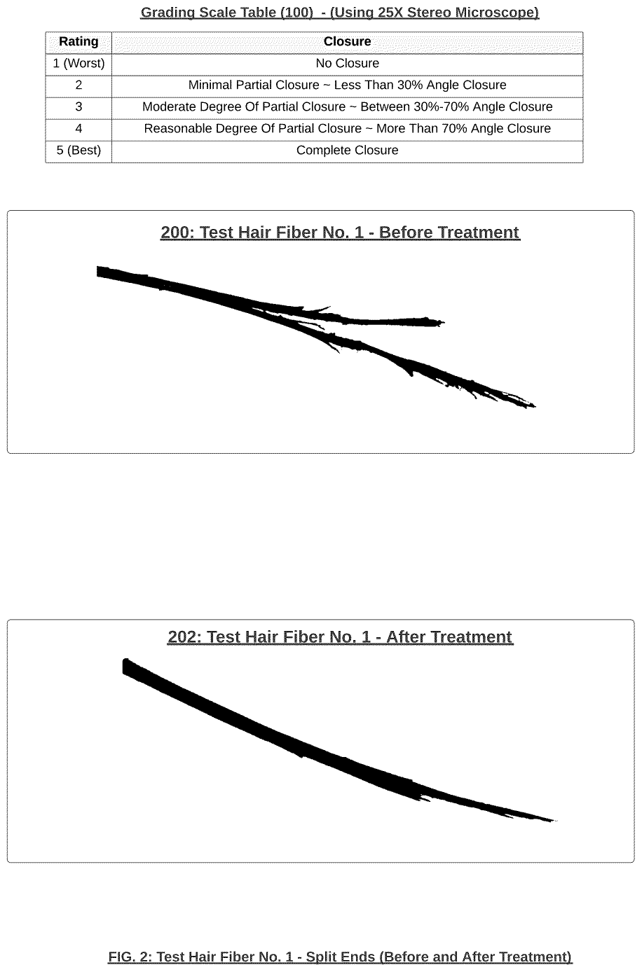

FIGS. 2, 3, 4, 5, and 6 collectively illustrate black and white conversions of the before-and-after microscopic images of hair test fibers 1-5 referenced above. In the interest of drawing brevity, images and figures for only the first five (5) fibers are shown graphically, but Table 2 provides the scores achieved for each and every fiber in the fifty (50) fiber test (i.e., include additional test fiber nos. 6-50).

The efficacy of the composition in improving the visual appearance of split ends and enhancing hair integrity after being subjected to the treatment is illustrated in FIGS. 2-6. Each figure presents comparative black and white versions of the images of the individual test hair fibers before and after application of the treatment, highlighting the degree of split-end closure and structural improvement achieved through the formulation. These figures provide quantifiable visual evidence supporting the claims of the invention, confirming that the composition successfully smooths and refines the ends of damaged hair fibers.

FIG. 2 displays black and white versions of the before-and-after microscopic images of Test Hair Fiber No. 1, illustrating the condition of the fiber prior to treatment and the improvements observed following application. The left-side image, labeled “Before Treatment (201),” reveals a distinct split end with visible separation between fiber strands. The right-side image, labeled “After Treatment (202),” demonstrates partial closure of the split end, indicating that the composition has smoothed the hair fiber and reduced the degree of visible damage. The alignment of the fiber strands has improved, creating a more polished and refined appearance.

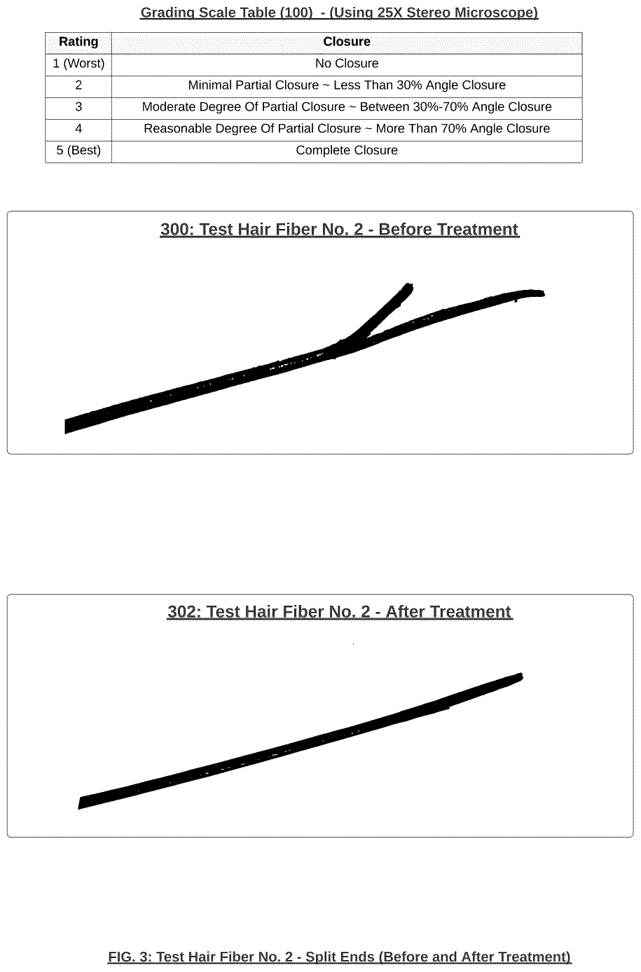

FIG. 3 continues the comparative analysis with black and white versions of the images of Test Hair Fiber No. 2, documenting its transformation following treatment. The left-side image, labeled “Before Treatment (301),” depicts a significantly frayed and separated split end, with multiple fiber strands splaying outward. The right-side image, labeled “After Treatment (302),” showcases a notable reduction in fiber separation, with the treated hair appearing smoother and more cohesive. The split ends exhibit visible realignment, with fiber strands drawn closer together, reducing the overall roughness and improving the aesthetic quality of the hair.

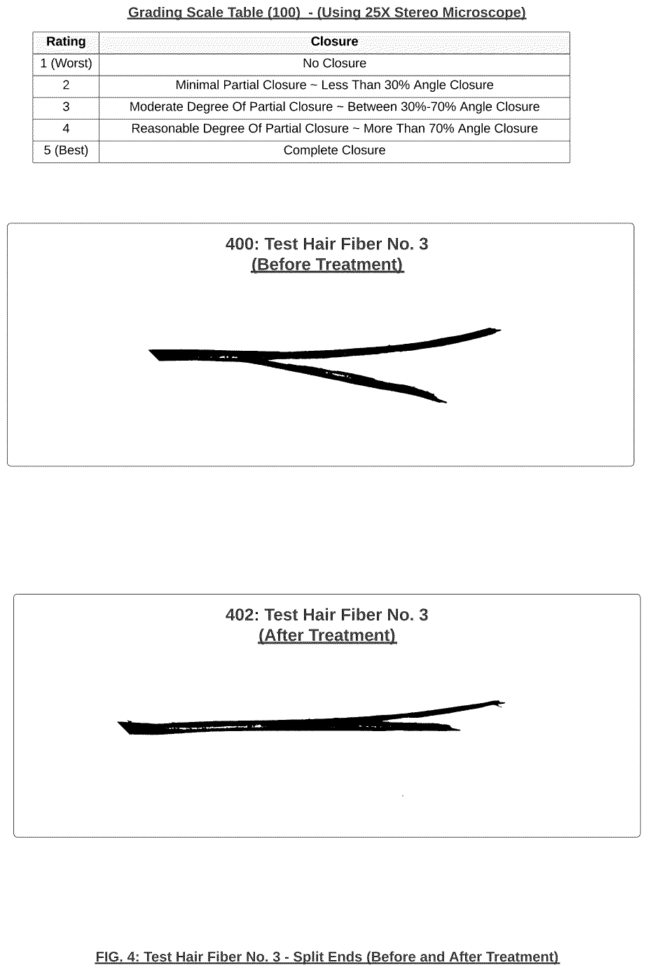

FIG. 4 presents black and white versions of the microscopic images of Test Hair Fiber No. 3 before and after application of the treatment. The left-side image, labeled “Before Treatment (401),” shows a damaged hair fiber with a severe split end, characterized by a wide and uneven opening. The right-side image, labeled “After Treatment (402),” reveals a significant improvement in the appearance of the split end, with the gap between the fiber strands visibly reduced. The treatment has sealed the hair fiber to a greater extent, resulting in a smoother, healthier-looking end.

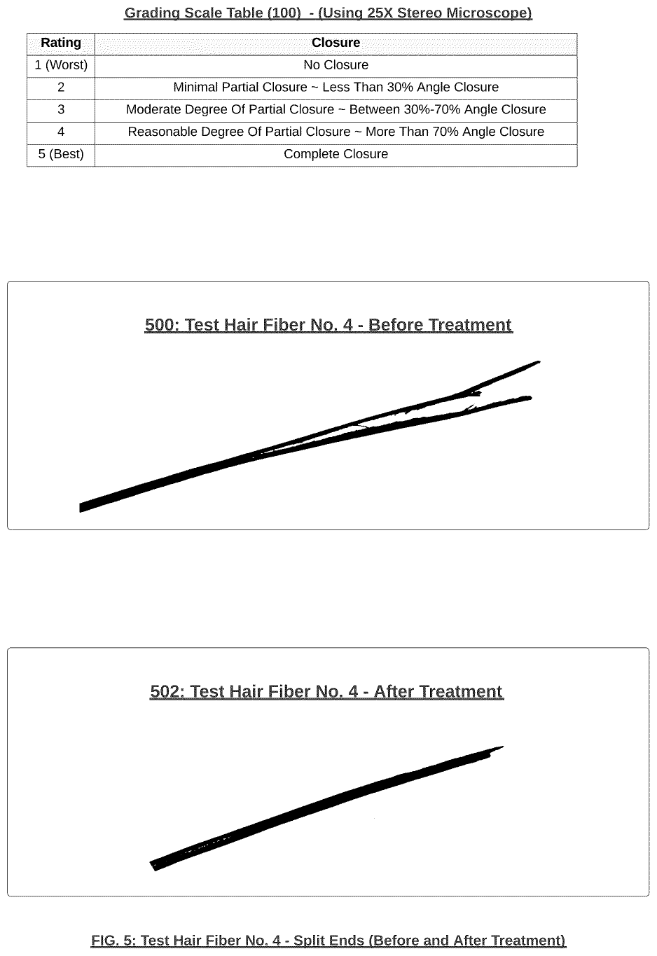

FIG. 5 provides additional comparative data, illustrating the black and white versions of the transformation of Test Hair Fiber No. 4 following treatment. The left-side image, labeled “Before Treatment (501),” depicts a damaged fiber with multiple split strands extending outward in different directions. The right-side image, labeled “After Treatment (502),” exhibits a pronounced improvement, with the split fibers drawn back into a more aligned, uniform structure. The treatment has visibly smoothed the fiber surface, reducing roughness and enhancing the overall integrity of the hair strand.

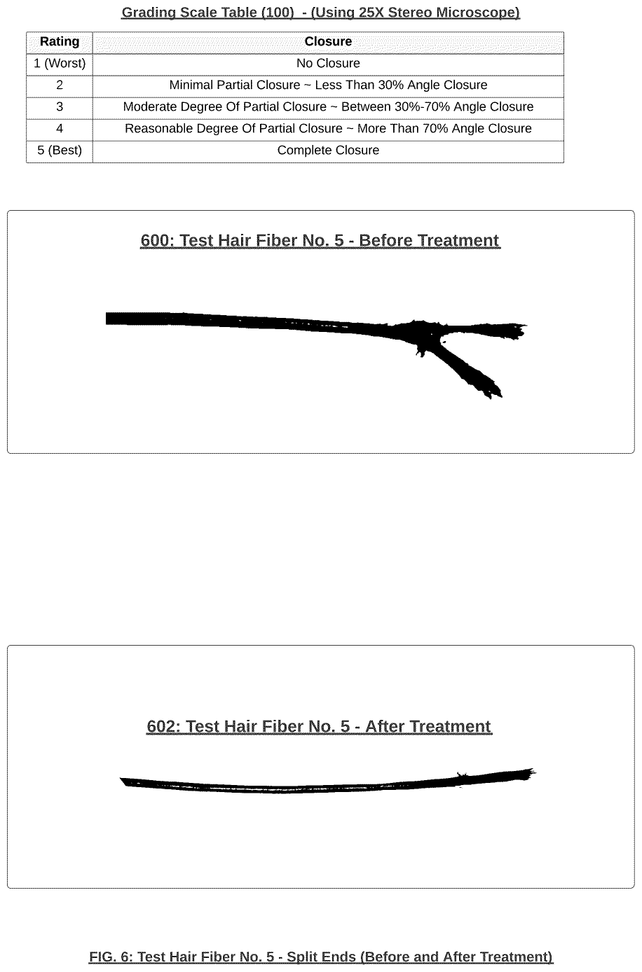

FIG. 6 completes the sequence of comparative black and white versions of microscopic images, documenting the results observed in Test Hair Fiber No. 5. The left-side image, labeled “Before Treatment (601),” illustrates a frayed and highly damaged hair fiber, with multiple separated strands forming an irregular, jagged structure. The right-side image, labeled “After Treatment (602),” demonstrates the effect of the composition in sealing and smoothing the fiber, significantly reducing the appearance of the split ends. The treated hair fiber exhibits a more cohesive, uniform structure, confirming the ability of the formulation to improve the visual appearance of damaged hair.

Collectively, FIGS. 2-6 serve as critical evidence supporting the effectiveness of the rinse-off hair treatment composition in improving the condition of split ends. The before-and-after images provide direct visual confirmation that the formulation reduces the degree of fiber separation, enhances smoothness, and refines the overall appearance of hair. These figures substantiate the claims that the composition delivers measurable improvements in split-end closure, reinforcing its role as a highly effective treatment for damaged hair. By documenting the transformations of multiple test hair fibers, these figures establish a consistent pattern of efficacy, further validating the ability of the formulation to address hair damage across different samples.

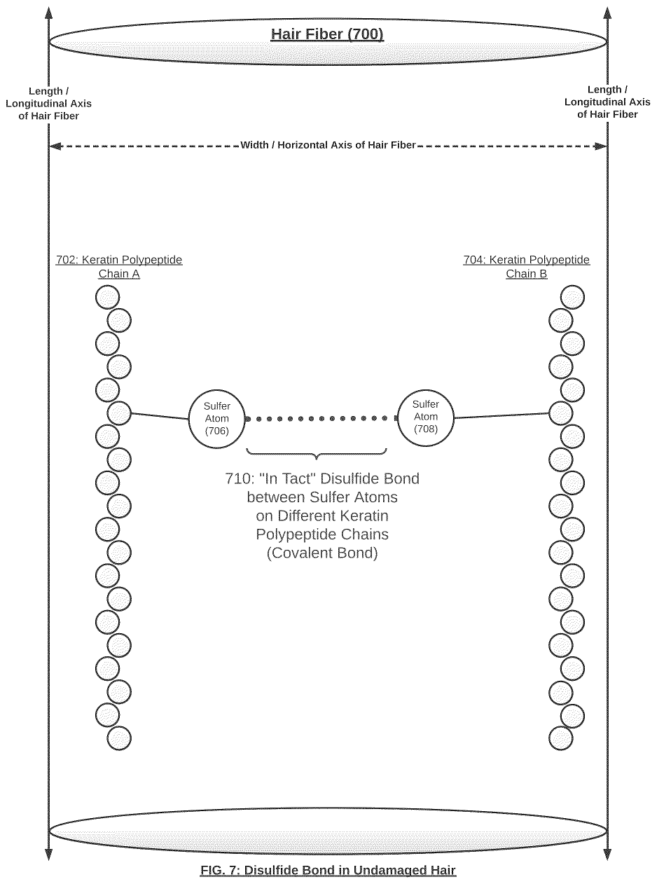

FIGS. 7, 8, 9, and 10 provide an illustrative molecular representation of the structural transformations that occur within hair fibers as a result of external damage and subsequent treatment with the innovative, rinse-off, hair-treatment, repair-mask composition disclosed herein. These figures visually depict the state of disulfide bonds, which play a crucial role in maintaining the strength, elasticity, and integrity of hair. The disulfide bonds, formed by the covalent bonding of sulfur atoms between cysteine residues in keratin polypeptide chains, are essential for holding the hair structure together. When these bonds are intact, the hair exhibits resilience, flexibility, and durability. However, when disulfide bonds are broken due to chemical processing, environmental stressors, or mechanical damage, the hair fiber weakens, leading to increased fragility, brittleness, and a greater susceptibility to split ends. These figures illustrate each stage of this structural breakdown and the process by which the treatment composition restores the bonds and strengthens the hair.

FIG. 7 presents a molecular-level depiction of an undamaged disulfide bond within a healthy hair fiber. This figure serves as a foundational reference, illustrating the strong and intact sulfur-sulfur covalent bond that connects two keratin polypeptide chains. The diagram is labeled “Undamaged Disulfide Bond (701)” and showcases the natural structure of keratin proteins within the cortex of the hair. Arrows point to the disulfide bond, emphasizing its role in maintaining the overall cohesion and strength of the fiber. This representation conveys the importance of intact disulfide bonds in preserving the mechanical and structural properties of hair, ensuring that it remains resistant to breakage and external damage. The diagram highlights how, under normal conditions, these molecular cross-links provide stability, allowing the keratin proteins to form a strong and flexible matrix that contributes to the hair's healthy appearance and durability. The visualization of this stable structure establishes a baseline for understanding the effects of disulfide bond degradation in subsequent figures.

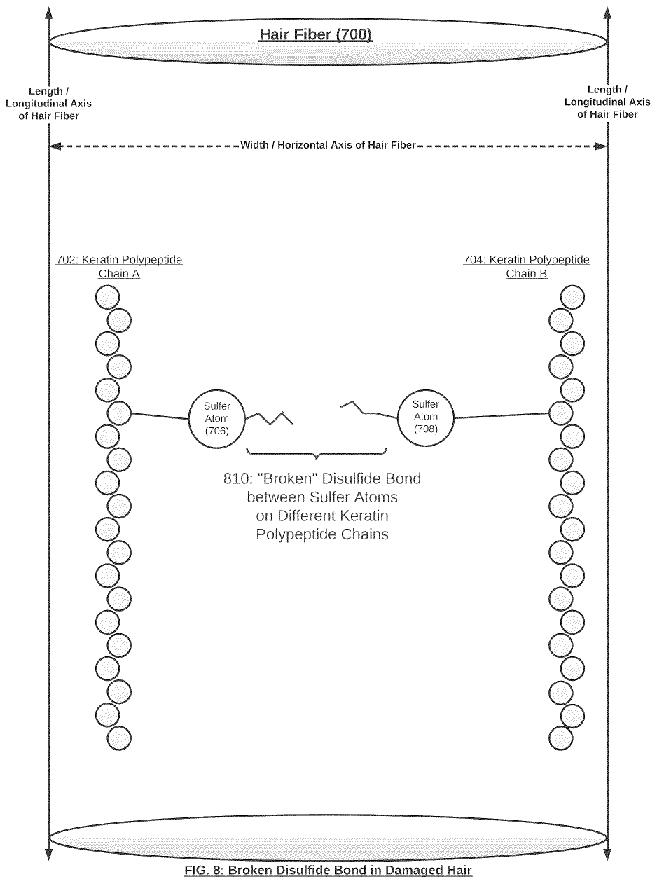

FIG. 8 illustrates the detrimental effects of external damage on the disulfide bonds within the hair fiber, depicting a scenario where these critical molecular connections have been broken. The diagram is labeled “Broken Disulfide Bond (801)” and shows the disruption of the sulfur-sulfur covalent bond, leading to the separation of keratin polypeptide chains. The figure provides a clear representation of how external aggressors such as bleaching, heat styling, UV exposure, environmental pollutants, and harsh chemical treatments sever these molecular linkages, resulting in structural instability. The arrows in this figure point to the sites of bond breakage, highlighting the areas where the molecular framework has been compromised. The depiction emphasizes that when disulfide bonds are fractured, the keratin matrix loses its structural integrity, leading to increased porosity, loss of elasticity, and reduced tensile strength. As a result, the hair becomes more prone to frizz, split ends, and overall deterioration. The illustration of the broken bond effectively communicates the underlying cause of hair damage and establishes the need for a treatment solution capable of repairing these critical molecular structures.

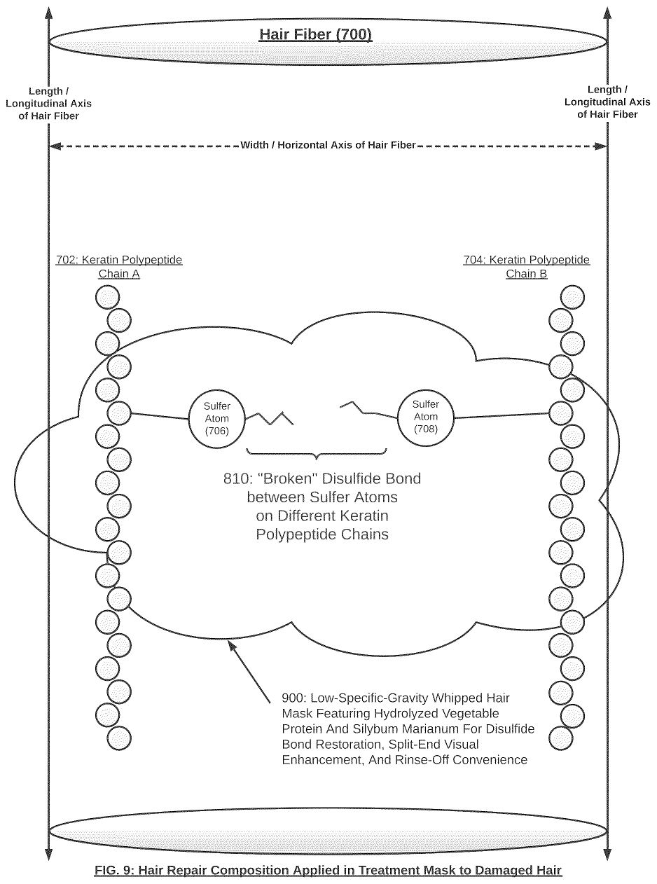

FIG. 9 demonstrates the application of the disclosed hair treatment composition to a damaged hair fiber, illustrating the process by which the formulation interacts with broken disulfide bonds to initiate the repair process. The diagram is labeled “Application of Hair Treatment Composition (901)” and depicts active ingredients penetrating the hair cortex and targeting the fractured keratin chains. The figure includes wavy lines representing the deposition of restorative agents, specifically highlighting the roles of Hydrolyzed Vegetable Protein PG-Propyl Silanetriol and Silybum Marianum Seed Extract. These ingredients work synergistically to facilitate molecular restructuring by reinforcing the protein framework and protecting against oxidative stress. The illustration also includes directional arrows indicating the movement of the treatment formulation within the hair fiber, showing how the composition actively seeks out and binds to damaged sites. This targeted interaction is essential for restoring cohesion between keratin chains, ensuring that the repaired bond is not only visually improved but also reinforced at a structural level. The depiction of this process conveys the precision and efficacy of the treatment, demonstrating how it penetrates the fiber to deliver lasting repair benefits.

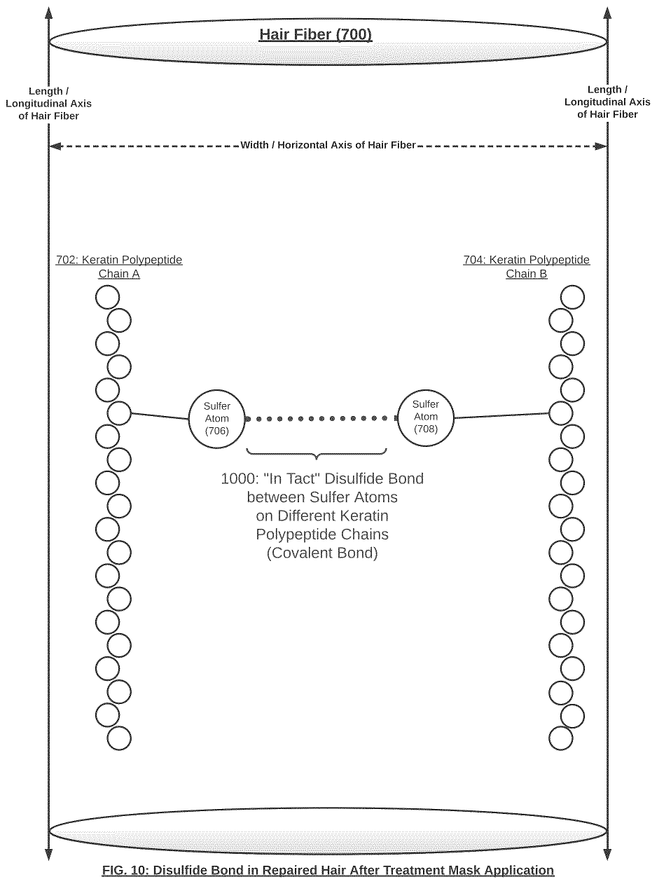

FIG. 10 presents the final stage of the molecular repair process, illustrating a fully restored disulfide bond following treatment with the composition. The diagram is labeled “Repaired Disulfide Bond (1001)” and shows the successful reformation of the sulfur-sulfur covalent bond, reconnecting the previously fractured keratin polypeptide chains. The figure highlights the stabilized molecular framework, emphasizing that the reestablishment of these bonds restores the internal strength, elasticity, and resilience of the hair fiber. Arrows in this figure point to the reformed disulfide bond, indicating that the hair has regained its structural integrity, allowing it to withstand future exposure to environmental and mechanical stressors. The illustration further emphasizes that the treatment not only facilitates bond reformation but also enhances the overall cohesion of the keratin matrix, preventing further breakage and deterioration. The visualization of this final stage reinforces the effectiveness of the composition in delivering tangible improvements in hair health, confirming that the repair process is both functional and enduring.

Together, FIGS. 7, 8, 9, and 10 provide a comprehensive molecular-level explanation of the damage-repair cycle within hair fibers. These figures illustrate the transition from a healthy, intact disulfide bond to a broken and structurally compromised state due to external aggressors. They further depict the targeted application of the hair treatment composition and the successful reformation of the disulfide bond, demonstrating the restorative properties of the formulation. By presenting a clear and detailed sequence of molecular transformations, these figures establish a strong scientific foundation for the claims of the invention, validating its ability to repair disulfide bonds, reinforce keratin structure, and improve overall hair integrity. This molecular repair mechanism directly contributes to the observed improvements in hair strength, elasticity, frizz reduction, and split-end appearance, providing both immediate and long-term benefits for users seeking to restore and maintain healthy hair.

Regarding disulfide bond testing, an extensive analysis of the ability of the inventive composition to restore disulfide bonds in chemically damaged hair was performed. Disulfide bonds are critical structural components within the keratin matrix of hair, providing strength, resilience, and elasticity. When hair undergoes chemical processing such as bleaching, coloring, or perming, these bonds break down, leading to weakened and fragile hair fibers. The purpose of this study was to evaluate the effectiveness of the conditioning mask in repairing these broken bonds and restoring the hair's structural integrity. The study used advanced Confocal Raman Spectroscopy, a non-destructive molecular analysis technique, to quantitatively measure the presence, distribution, and alignment of disulfide bonds within the hair fiber before and after treatment.

The hair samples used in the study were carefully selected to represent different levels of damage. Virgin, untreated Piedmont Blond hair tresses were used as a control group to establish baseline measurements of intact disulfide bonds. The second test group consisted of hair tresses that had undergone three rounds of bleaching with a nine percent peroxide developer, simulating extreme chemical damage. The third group consisted of bleached hair tresses that were subsequently treated with the inventive mask for five consecutive applications to assess the product's ability to restore the lost disulfide bonds. This experimental design ensured that the findings would accurately reflect the degree of molecular repair provided by the hair mask.

Each hair tress underwent a standardized washing and treatment process to ensure consistency across samples. The hair tresses were first washed using a non-conditioning shampoo at a concentration of ten percent by weight of the tress. This step removed any residual oils or contaminants that could interfere with the accuracy of the molecular analysis. Following the washing process, the inventive composition was applied at a concentration of ten percent by weight of the tress, ensuring that all hair fibers received an even coating of the treatment. The mask was then massaged into the hair for thirty seconds to enhance penetration and left on for fifteen minutes to allow the active ingredients to interact with the damaged hair structure. After the treatment period, the tresses were rinsed thoroughly with warm water at forty degrees Celsius for thirty seconds. The hair was then blow-dried at a low heat setting for five minutes. This entire treatment process was repeated five times to evaluate the cumulative effects of multiple applications.

Confocal Raman Spectroscopy was used to measure the presence and alignment of disulfide bonds in each test group. This technique allowed researchers to obtain highly detailed molecular-level data on the structural composition of the hair. The Raman spectral analysis focused on the disulfide bond region at 516 cm⁻¹, which was normalized against the amide I band at 1655 cm⁻¹ to provide a precise comparison of bond recovery. Spectral measurements were taken at multiple depths within the hair fiber, with data collected at five-micron intervals up to a depth of twenty-five microns. This approach enabled researchers to assess both surface-level and internal bond restoration, providing a comprehensive understanding of the effectiveness of the treatment.

The study found that untreated virgin hair exhibited the highest levels of disulfide bond content, confirming that intact, healthy hair has a well-organized keratin structure. In contrast, the chemically bleached hair group showed a significant loss of disulfide bonds, with approximately fifty-eight percent of the bonds being degraded due to chemical damage. The application of the treatment mask resulted in a remarkable improvement, with seventy percent (70%) of the lost disulfide bonds being restored after five (5) consecutive treatments. This finding demonstrated that the conditioning mask was capable of not only improving the surface-level appearance of the hair but also reinforcing the internal structure, effectively reversing the damage caused by bleaching.

Hyperspectral Raman imaging provided further confirmation of the bond restoration process. Color-mapped data visually represented the distribution of disulfide bonds within the hair fiber, with red areas indicating a high concentration of intact bonds and blue areas indicating areas of damage or bond loss. The untreated virgin hair displayed a predominantly red pattern, signifying a high level of disulfide bond integrity. The bleached hair group exhibited extensive blue regions, highlighting the severe bond degradation resulting from chemical processing. The treated hair samples, however, showed a significant shift back toward red, indicating that the inventive treatment mask successfully replenished the lost bonds and restored the structural integrity of the hair.

In addition to quantifying disulfide bond content, the study also examined the molecular conformation of the repaired bonds. Disulfide bonds can adopt different structural conformations depending on their alignment and integrity. The study measured the ratio of properly aligned disulfide bonds, referred to as the GGG conformation, versus the misaligned bonds, referred to as the TGT conformation. Virgin hair exhibited the lowest amount of TGT conformation, indicating a highly organized keratin matrix. Bleached hair had a significantly higher TGT content, confirming structural misalignment and protein disruption. After treatment with the inventive treatment mask, the TGT/GGG ratio shifted toward a more organized structure, suggesting that the product not only restored bond quantity but also improved bond alignment, bringing the hair closer to its natural, undamaged state.

Statistical analysis was performed to validate the findings. A one-way ANOVA test confirmed that the differences in disulfide bond content between the bleached and treated samples were statistically significant, with a p-value of less than 0.05. This statistical threshold demonstrated that the bond restoration observed in the treated samples was not due to random variation but rather a direct result of the product's reparative action. Further statistical comparisons showed that the treated hair fibers had disulfide bond levels that were significantly closer to virgin hair than to bleached hair, confirming the effectiveness of the treatment.

The study's findings support the conclusion that the treatment mask provides substantial molecular repair to chemically damaged hair. The treatment was shown to restore seventy percent of lost disulfide bonds after five consecutive applications, reinforcing the internal keratin network and improving the overall structural integrity of the hair fiber. The product also improved bond alignment, reducing the presence of improperly formed disulfide linkages that are characteristic of damaged hair. These results suggest that the hair mask is highly effective for individuals with chemically processed hair who seek to restore strength, elasticity, and resilience to their hair.

Overall, this study provides strong scientific evidence that the inventive composition is an effective treatment mask for repairing chemically damaged hair by restoring disulfide bonds, reinforcing keratin structure, and improving molecular alignment. These findings establish the formulation as a highly beneficial treatment for strengthening and protecting hair fibers, offering both immediate and cumulative repair benefits.

Anti-frizz testing was performed to evaluate the ability of the inventive rinse-off composition to control frizz and maintain smoothness under high humidity conditions. The study was designed to compare the performance of the inventive rinse-off composition against a control treatment consisting of a 15% SLES solution, which is a non-conditioning shampoo. The primary objective was to quantify the reduction in frizz over time and assess whether the treatment provided sustained anti-frizz benefits.

The study tested multi-ethnic frizzy hair, specifically curly type 3B hair, which is highly susceptible to humidity-induced swelling and frizz. The hair tresses used in the study measured approximately eight inches in length and four grams in weight. To ensure statistically significant results, eight hair tresses were tested per treatment group, providing a robust data set for analysis.

The frizz control experiment followed a standardized process. Initially, all hair tresses were washed using a 15% SLES solution, which is a non-conditioning sulfate-based shampoo, at a concentration of ten percent by weight of the tress. This step was necessary to remove any residual conditioning agents and expose the natural frizz-prone characteristics of the hair. After washing, the tresses were left overnight at 20% relative humidity and 22 degrees Celsius in a controlled environment to standardize moisture levels before treatment.

To establish a maximum frizz baseline, the tresses were then combed to create a state of maximum fiber misalignment before treatment. Baseline images were captured to document the initial frizz levels. Each tress was then treated with either the inventive rinse-off composition or the control solution and subsequently exposed to high humidity conditions of 80% relative humidity for 24 hours. Frizz levels were measured at three key time points: immediately after treatment, after eight hours, and after twenty-four hours.

To simulate real-world product application, the inventive rinse-off composition was applied using a standardized method. The mask was applied at twenty percent by weight of the tress, equivalent to 0.2 milliliters per gram of dry hair, or 0.8 milliliters per four-gram tress. The composition was massaged into the hair for thirty seconds, left on for ten minutes, and then rinsed for thirty seconds. The control group followed the same process using the SLES solution, which did not contain conditioning ingredients. After treatment, the tresses were re-equilibrated overnight at 20% relative humidity before being placed in an 80% relative humidity chamber, where they remained for the duration of the study.

To objectively measure frizz reduction, digital images were taken at zero, eight, and twenty-four hours using custom image analysis software to quantify the frizz volume of each hair tress in pixels. The percentage reduction in frizz was calculated by comparing the frizz pixel count of the treated samples against the maximum frizz state. This provided a precise and reproducible method for evaluating the efficacy of the treatment.

The results of the study demonstrated that the inventive rinse-off composition significantly reduced frizz at all measured time points compared to the control treatment. Statistical analysis was conducted using Statistica and JMP analytical software, with a Student's t-test at a 95% confidence level applied to determine significance. Immediately after treatment, the inventive rinse-off composition reduced frizz by 63.24%, whereas the control treatment achieved only a 32.06% reduction. This difference was statistically significant, confirming that the inventive rinse-off composition provided immediate and superior frizz control.

At the eight-hour mark, the inventive rinse-off composition continued to demonstrate strong frizz control, maintaining a 61.35% reduction compared to the control treatment, which reduced frizz by only 37.08%. The control group showed more rapid frizz reversion over time, while the tresses treated with the inventive rinse-off composition retained their smooth appearance. After twenty-four hours of exposure to 80% humidity, the inventive rinse-off composition maintained a 61.20% reduction in frizz, compared to 37.38% for the control treatment. These results indicated that the inventive rinse-off composition provided sustained frizz resistance over an extended period, effectively preventing humidity-induced fiber misalignment.

Frizz occurs when hair absorbs moisture from the surrounding environment, causing the cuticle to lift and fiber misalignment to increase. This effect is particularly pronounced in curly and textured hair types, which have higher porosity and are more prone to humidity-induced expansion. The findings from this study demonstrated that the inventive rinse-off composition effectively prevents frizz by forming a humidity-resistant barrier on the hair surface, which reduces water uptake and maintains fiber alignment.

The difference in performance between the inventive rinse-off composition and the control group was substantial at all time points, indicating that the conditioning mask provided both immediate and long-lasting frizz control. The ability of the composition to maintain a 61% reduction in frizz after twenty-four hours suggests that its active ingredients played a critical role in protecting the hair against external humidity.

The high efficacy of the inventive rinse-off composition is due to its dual-action frizz control mechanism. The formulation smooths the cuticle layer, reducing the gaps that allow moisture to penetrate while simultaneously forming a protective layer that limits water absorption. This prevents the cuticle from swelling and becoming misaligned.

The results of this study provide strong scientific validation that the inventive rinse-off composition is highly effective in controlling frizz and maintaining hair smoothness under high-humidity conditions. The composition demonstrated a statistically significant frizz reduction of 61% compared to the control's 37% reduction, with effects lasting at least twenty-four hours. This confirms that the inventive rinse-off composition is not only superior to a non-conditioning control but also provides long-term frizz resistance in challenging humidity conditions.

The findings suggest that the inventive rinse-off composition is an optimal solution for individuals with frizz-prone hair, particularly those with curly, wavy, or chemically processed hair types. The ability of the composition to provide long-lasting frizz reduction makes it well-suited for consumers looking for humidity protection and enhanced hair manageability. Given its strong performance in controlled lab testing, it is expected that real-world application would provide similarly effective results.

The study also indicates that multiple applications may provide cumulative benefits, potentially increasing the level of frizz protection with repeated use. Future research could explore the composition's performance under different humidity levels, hair porosities, and application frequencies, as well as compare its efficacy against competitor anti-frizz treatments.

The results of this testing confirm that the inventive rinse-off composition is a scientifically proven anti-frizz solution that provides immediate, measurable, and long-lasting smoothness in high humidity conditions. The study's rigorous testing and statistical analysis establish it as an effective treatment for maintaining frizz control, ensuring that hair remains smooth and manageable even in challenging environmental conditions.

Regarding anti-breakage, the ability of the inventive rinse-off composition to protect hair fibers from breakage caused by repeated grooming was evaluated. The tests were conducted under controlled conditions using European medium brown hair tresses that had been chemically bleached with a 9% hydrogen peroxide solution. The study assessed the number of broken fibers after extensive mechanical stress, simulating real-world brushing and combing routines. The results demonstrated that the inventive rinse-off composition significantly reduced hair breakage compared to untreated control samples, confirming its effectiveness in reinforcing hair strength and preventing damage.

The study followed a standardized experimental design to ensure accurate and reproducible results. Hair tresses measuring eight inches in length and one inch in width were first cleansed with a 15% SLES (sodium lauryl ether sulfate) solution, a non-conditioning detergent, to remove any residual products and normalize the hair's baseline condition. Each tress was then subjected to either the control treatment, which consisted of repeated washing with SLES alone, or the inventive rinse-off composition, which was applied in a measured amount, left on for a specific duration, and rinsed under controlled conditions.

The hair tresses were then exposed to a mechanical stress test using an automated repeated grooming apparatus. This device, a custom-built automated grooming simulator, used a rotating drum with mounted combs and brushes that repeatedly passed through the hair tresses. Each full revolution of the drum resulted in four strokes through each tress, and the process was repeated for a total of 2,000 strokes per sample. Broken fibers were collected on designated collection plates after every 200 strokes to document the progression of damage over time. The total number of broken fibers per tress was recorded and statistically analyzed.

Two separate tests were conducted to evaluate the effect of different application durations of the inventive rinse-off composition. In the three-minute application test, the composition was applied at twenty percent by weight of the tress, left on for three minutes, and then rinsed for thirty seconds with water at forty degrees Celsius at a controlled flow rate of one gallon per minute. The tresses were allowed to air dry overnight at sixty percent relative humidity before testing. In the ten-minute application test, the same procedure was followed, except the product was left on for ten minutes instead of three.

The results of the anti-breakage tests were analyzed using box-and-whisker plots generated with Statistica and JMP statistical software, with a Student's t-test at a ninety-five percent confidence level used to determine statistical significance. The three-minute application test showed a seventy-seven percent reduction in hair breakage when compared to the control treatment. Hair tresses washed with SLES alone in the control group experienced an average of forty-three broken fibers per tress after two thousand strokes, while tresses treated with the inventive rinse-off composition for three minutes experienced only ten broken fibers per tress. This statistically significant reduction demonstrated that even a short three-minute application substantially reinforced hair fibers and minimized breakage from mechanical stress.

In the ten-minute application test, the effect was even more pronounced, with an eighty-three percent reduction in breakage. Hair tresses subjected to the control treatment had an average of forty-six broken fibers per tress, while those treated with the inventive rinse-off composition had only eight broken fibers per tress. The additional leave-in time allowed for greater penetration of active ingredients, leading to a more substantial reduction in breakage. The findings suggest that longer application times provide enhanced protection against mechanical damage, making the treatment adaptable for different levels of hair damage and user preferences.

The success of the inventive rinse-off composition in preventing breakage is attributed to its multi-functional conditioning and strengthening ingredients. Hydrolyzed Vegetable Protein PG-Propyl Silanetriol penetrates the hair shaft and strengthens the keratin structure, reinforcing weak areas that are prone to snapping. Silybum Marianum Seed Extract provides antioxidant protection and structural reinforcement, preventing further degradation of chemically treated hair.

The study confirmed that the inventive rinse-off composition significantly improves hair resilience, making it an effective solution for individuals experiencing excessive breakage due to chemical processing, heat styling, or frequent brushing and combing. The ability of the treatment to reduce breakage by up to eighty-three percent demonstrates its potential for long-term hair repair and maintenance, offering both immediate and cumulative benefits with repeated use. The research further indicates that both short and extended application times provide measurable improvements, allowing consumers to tailor the treatment to their specific needs.

The findings suggest that continued use of the inventive rinse-off composition could provide increasing protection over time, reinforcing the hair structure and preventing future damage. The data from this study strongly supports the efficacy of the inventive rinse-off composition in reducing hair breakage, making it an advanced and scientifically validated solution for reinforcing and protecting weakened hair fibers.

Regarding wet combing testing, the ability of the inventive rinse-off composition to reduce combing friction in bleached and chemically treated hair, thereby improving manageability, reducing tangling, and minimizing mechanical stress on hair fibers, was evaluated. The study assessed the force required to comb through wet hair tresses treated with the inventive rinse-off composition compared to a control group that was only washed with a 15% sodium lauryl ether sulfate (SLES) solution, which served as a non-conditioning detergent control. The tests measured combing force using an Instron tensile tester, an advanced instrument that quantitatively evaluates frictional resistance when a comb passes through treated hair strands.

The experiments were performed on European medium brown hair tresses that had been bleached using a 6% hydrogen peroxide solution, simulating hair that is highly porous, damaged, and prone to tangling. The hair tresses were standardized at three grams in weight, eight inches in length, and one inch in width, ensuring that all test samples had consistent characteristics. Before applying any treatment, the tresses were thoroughly cleansed with SLES shampoo to remove any residual products and restore their natural baseline condition.

The testing protocol involved two separate application time periods: one study evaluated a three-minute application of the inventive rinse-off composition, while another tested a ten-minute application. The three-minute application process involved applying the treatment at 20% by weight of the tress, massaging it in for 30 seconds, allowing it to sit for three minutes, and rinsing it with 40° C. water for 30 seconds at a controlled flow rate of one gallon per minute. The ten-minute application process followed the same methodology but extended the leave-in time to ten minutes before rinsing. After treatment, the hair tresses were immediately tested in their wet state using the Instron tensile tester. Each tress underwent six combing strokes, with a total of eight replicate tresses per treatment group, ensuring that the data had statistical relevance.

The primary goal of the study was to determine whether the inventive rinse-off composition could significantly reduce combing force, thereby improving hair smoothness, detangling properties, and ease of styling. The data was analyzed using box-and-whisker plots generated with Statistica and JMP analytical software, and statistical significance was determined using a Student's t-test at a 95% confidence level.

The results demonstrated that the inventive rinse-off composition produced a dramatic reduction in combing force compared to the control treatment. In the three-minute application study, the control group required an average combing force of 157.1 grams-force (gf), whereas the tresses treated with the inventive rinse-off composition required only 10.02 gf. This represented a 94% reduction in combing force, confirming that the composition provided substantial detangling benefits and lubrication, significantly lowering resistance during combing.

In the ten-minute application study, the results were similarly impressive. The control group required an average combing force of 117.36 gf, whereas the tresses treated with the inventive rinse-off composition required only 11.74 gf. This translated to a 90% reduction in combing force, further validating the effectiveness of the formulation in reducing mechanical friction and improving manageability.

The drastic reduction in combing force achieved by the inventive rinse-off composition is attributed to its advanced conditioning and smoothing agents, which improve fiber alignment, reduce inter-fiber friction, and coat the hair strands with a lightweight lubricating layer. The inclusion of Hydrolyzed Vegetable Protein PG-Propyl Silanetriol strengthens the cuticle, further minimizing friction and reducing fiber breakage during grooming.

The difference in combing force between the three-minute and ten-minute application times suggests that even short-term exposure to the inventive rinse-off composition provides significant improvements in hair smoothness and detangling, while longer leave-in times allow for additional penetration and reinforcement of the cuticle structure. The results indicate that the product is highly effective regardless of the duration of application, making it adaptable for both quick-use and deep-conditioning treatments.

The study confirmed that the inventive rinse-off composition dramatically improves wet combing properties, making hair easier to detangle and reducing mechanical stress that can lead to breakage. The reduction in combing force by up to 94% compared to the control group provides strong evidence that the formulation significantly enhances hair lubrication, reduces tangles, and improves overall manageability. This makes it an excellent solution for individuals with chemically treated, porous, and damaged hair that is prone to knots and breakage. The research also suggests that continued use of the inventive rinse-off composition could provide cumulative benefits, as repeated applications would progressively reinforce the cuticle and maintain low combing resistance over time.

In summary, this patent application for the innovative for the Low-Specific-Gravity Whipped Hair Mask describes an advanced formulation designed to restore disulfide bonds, reduce hair breakage, and improve the appearance of split ends. The formulation features Hydrolyzed Vegetable Protein PG-Propyl Silanetriol, which penetrates the hair cortex and actively repairs broken disulfide bonds, reinforcing the keratin structure and enhancing tensile strength. Additionally, the inclusion of Silybum Marianum Seed Extract provides antioxidant protection, neutralizing oxidative stress that contributes to hair degradation while preserving the restored bonds. Unlike traditional deep-conditioning treatments that rely on heavy formulas, this hair mask incorporates a lightweight, whipped texture with a low-specific-gravity composition, which allows for even application, superior absorption, and effortless rinsability. This innovative formulation ensures that the treatment delivers intense repair without weighing the hair down, making it suitable for all hair types, including fine and textured hair.

The scientific efficacy of the formulation has been extensively validated through multiple performance-based studies. Tests conducted using confocal Raman spectroscopy confirmed that a single application of the mask restored approximately 20% of lost disulfide bonds, while five consecutive applications resulted in a 70% restoration. These findings indicate that the mask provides deep structural repair, reinforcing the hair's integrity and making it more resistant to damage over time. The disulfide bond repair was observed not only on the surface but also within the cortex, demonstrating the deep-penetrating ability of the active ingredients. Additionally, the treatment altered the disulfide bond conformation, reducing the presence of weaker bond structures and further strengthening the keratin network.

The product's ability to enhance hair manageability was demonstrated through wet combing resistance tests. A three-minute application of the mask resulted in a 94% reduction in wet combing force compared to the control, confirming a significant improvement in detangling and lubrication. A longer, ten-minute treatment time further decreased combing force, suggesting that the formulation provides enhanced conditioning benefits with extended use. These findings indicate that the mask effectively smooths the hair surface, reducing friction and making it easier to style.

In addition to improving manageability, the formulation has been shown to significantly reduce hair breakage. Repeated grooming tests demonstrated that hair treated with the mask experienced far fewer broken fibers compared to untreated hair. After two thousand brush strokes, hair treated with the mask showed a substantial reduction in breakage, confirming that the product protects against mechanical stress. The longer, ten-minute treatment resulted in an even greater reduction in broken fibers, reinforcing the claim that extended application enhances the hair's structural integrity.

The formulation's impact on split ends was also evaluated through imaging and grading studies. Results showed that the treatment significantly improved the alignment of split fibers, with some achieving complete or near-complete closure. A five-point grading scale was used to assess the degree of closure, and the findings confirmed that the mask effectively smooths and seals split ends, making the hair appear healthier and more polished. This effect is particularly beneficial for individuals looking to maintain hair length without frequent trims.

Another critical benefit of the formulation is its ability to control frizz, particularly in high-humidity conditions. Tests conducted in an 80% relative humidity environment demonstrated a measurable reduction in frizz immediately after application, with the effect lasting up to 24 hours. The mask helped maintain the hair's structure, preventing excessive moisture absorption that can lead to swelling and frizz. This result confirms that the formulation enhances moisture resistance and improves cuticle smoothness, providing long-lasting frizz control.

Overall, the test results strongly support the claims made in this patent application. The Low-Specific-Gravity Whipped Hair Mask is not merely a superficial conditioning treatment but a scientifically validated solution for repairing hair at a molecular level. The formulation effectively restores disulfide bonds, reduces breakage, enhances manageability, seals split ends, and controls frizz. These findings establish the product as a breakthrough in hair repair technology, offering a highly effective and convenient alternative to traditional conditioning treatments. Its ability to deliver both immediate and long-term improvements in hair strength and appearance sets a new standard in the hair care industry.

A skilled artisan, upon reviewing the disclosure, will appreciate that there are numerous alternatives, modifications, combinations, and customizations that can be made to the systems and methods described herein.

In particular, the compositions and methods described in this application for the Low-Specific-Gravity Whipped Hair Mask allow for a wide range of alternatives, modifications, combinations, and customizations that remain within the spirit and scope of the disclosure. Various aspects of the formulation, including active ingredients, secondary conditioning agents, delivery methods, and treatment protocols, can be adjusted to meet different performance objectives, hair types, and market demands while preserving the core scientific principles of the invention.

One alternative involves adjusting the concentration of Hydrolyzed Vegetable Protein PG-Propyl Silanetriol, which is the primary disulfide bond-repairing agent. In the disclosed embodiment, this ingredient is included at approximately 0.175% by total weight, but effective concentrations can range from 0.05% to 2.00%. Lower concentrations (0.05% to 0.20%) may be used in formulations designed for daily or lightweight conditioning treatments, while higher concentrations (0.50% to 2.00%) may be included in intensive repair masks aimed at heavily processed or chemically treated hair. Increasing the protein content enhances structural reinforcement, but excessive amounts should be avoided to prevent hair rigidity or protein overload.

Similarly, Silybum Marianum Seed Extract, an antioxidant and protective agent, is present in a preferred embodiment at 0.050% but can be adjusted within a range of 0.025% to 1.00%. Lower concentrations may be sufficient when combined with other antioxidant agents such as green tea extract, vitamin E, or ferulic acid, whereas higher concentrations may be incorporated in formulations specifically targeting oxidative damage and UV protection.

Hydration-enhancing ingredients, such as Sodium Hyaluronate derivatives, can also be modified to suit different hair needs. Sodium Hyaluronate is included at approximately 0.0025% in one embodiment, with an effective range of 0.001% to 0.50%. Hydroxypropyltrimonium Hyaluronate, which provides both hydration and cuticle-smoothing benefits, can range from 0.001% to 0.30%, while Hydrolyzed Sodium Hyaluronate, a smaller molecular weight variant designed for deeper penetration, can be included at 0.0005% to 0.20%. These variations allow for customization based on desired moisture retention, hair porosity, and compatibility with other ingredients.

Alternative conditioning agents may also be incorporated to enhance the formula's performance. While the disclosed composition includes Dimethicone and Amodimethicone in carefully controlled ratios, alternative lightweight conditioning polymers such as Polyquaternium Compounds, Behentrimonium Chloride, or Cationic Guar Gum could be used potentially to modify the sensory feel and deposition characteristics. These alternatives would allow for tailoring to different hair types, particularly for consumers seeking silicone-free or sulfate-free options.

The aerated, whipped texture of the composition is achieved through high-shear mixing techniques and structuring agents like Cetearyl Alcohol, Cetyl Hydroxyethylcellulose, and Xanthan Gum. However, the specific gravity of the formulation could be adjusted further, with a target range of approximately 0.85 to 0.95, allowing for variations in density and spreadability. This could be particularly beneficial for adapting the formulation for different application methods, such as sprayable treatments, mousse-like foams, or thicker, richer masks.

The formulation can also be combined with additional reparative ingredients for enhanced efficacy. Peptide-based keratin derivatives, plant-derived amino acids, or cystine-based compounds could be introduced to further support disulfide bond repair and reinforcement. Customizations could include alternative botanical extracts, such as rosemary or bamboo extract, to provide added scalp benefits, or ceramides to improve cuticle integrity and lipid balance.

In terms of application methods, the treatment protocol could be adapted for various exposure times and hair conditions. The disclosed method involves a 15-minute application for maximum benefit, but alternative regimens could include a rapid 5-minute treatment for consumers seeking faster results, or an overnight leave-in version with adjusted emollient levels for deep penetration. The formulation could also be optimized for professional salon use, where heat activation or extended processing times may enhance penetration and efficacy.

Furthermore, the mask's composition can be customized to cater to specific hair types and textures. For fine or low-porosity hair, a lower concentration of conditioning agents combined with an emphasis on lightweight hydration and structural reinforcement would be ideal. For coarse, highly porous, or chemically processed hair, a richer formula with additional lipid-based emollients such as shea butter, argan oil, or coconut-derived esters could enhance softness and manageability.

The formulation also allows for modular ingredient swaps based on consumer preferences or regulatory considerations. For example, to cater to a clean beauty market segment, synthetic silicones could be replaced with plant-derived alternatives such as broccoli seed oil, which mimics the smoothing properties of traditional silicones. Similarly, preservatives could be adjusted based on regional requirements, replacing traditional parabens with naturally derived antimicrobial agents like fermented radish root extract.

To further expand the product line, the formulation could be adapted into a multi-step system, including a pre-treatment bond-building spray for deeper penetration, an in-shower conditioning mask, and a post-treatment sealing serum to prolong the effects. Additionally, variations of the mask with added color-protecting agents, UV filters, or thermal protection elements could be developed to address specific consumer concerns.

In summary, the Low-Specific-Gravity Whipped Hair Mask is highly adaptable, with numerous potential modifications that fall within the scope of the invention. Adjustments in active ingredient concentrations, alternative conditioning agents, formulation textures, and application protocols allow for extensive customization. These variations enable the formulation to be tailored for different hair types, consumer preferences, and market trends while preserving the core benefits of disulfide bond repair, hydration, frizz control, and lightweight conditioning. All are within the spirit and scope of the present disclosure.

Although the present technology has been described based on what is currently considered the most practical and preferred implementations, it is to be understood that this detail is only for that purpose and this disclosure is not limited to the sample descriptions and implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A rinse-off, low-specific-gravity, hair-repair treatment composition comprising:
a therapeutically effective amount of a Hydrolyzed Vegetable Protein-Silicone Hybrid, wherein the Hydrolyzed Vegetable Protein-Silicone Hybrid comprises Hydrolyzed Vegetable Protein PG-Propyl Silanetriol and is configured to penetrate hair fibers to restore broken disulfide bonds and reinforce keratin structures;
a therapeutically effective amount of a botanical antioxidant, wherein the botanical antioxidant comprises Silybum Marianum Seed Extract and is configured to neutralize oxidative stress and protect the restored disulfide bonds from further degradation;
wherein the rinse-off hair treatment composition has a specific gravity below about 0.90, forming a low-density, aerated, and easily spreadable formulation that enhances deposition and absorption while maintaining a lightweight feel;
wherein when the rinse-off hair treatment composition is applied to hair, left for at least 10 minutes, and rinsed, the rinse-off hair treatment composition restores at least about 30% of lost disulfide bonds and improves the visual appearance of at least about 60% of split ends, with at least about 40% of the split ends achieving complete closure; and
wherein the synergistic combination of the Hydrolyzed Vegetable Protein-Silicone Hybrid and the botanical antioxidant improves the visual appearance of split ends by reinforcing the hair fiber structure at the molecular level, reducing cortical fragmentation, and enhancing cuticle alignment, resulting in smoother, healthier-looking hair.

2. The composition of claim 1, wherein the Hydrolyzed Vegetable Protein-Silicone Hybrid is present in an amount ranging from about 0.05% to about 2.00% by total weight of the composition, and the botanical antioxidant is present in an amount ranging from about 0.025% to about 1.00% by total weight of the composition.

3. The composition of claim 2, wherein the Hydrolyzed Vegetable Protein-Silicone Hybrid is present in an amount of at least about 0.175% by total weight of the composition.

4. The composition of claim 3, wherein the botanical antioxidant is present in an amount of at least about 0.050% by total weight of the composition.

5. The composition of claim 4, wherein the composition, when applied to hair, left for at least 10 minutes, and rinsed, restores at least about 32% of lost disulfide bonds.

6. The composition of claim 5, wherein the composition, when applied to hair, left for at least 10 minutes, and rinsed, improves the visual appearance of at least about 68% of split ends, with at least about 46% of the split ends achieving complete closure.

7. The composition of claim 6, wherein the composition is configured to reduce hair breakage by at least about 80% after 2,000 brush strokes in repeated grooming tests.

8. The composition of claim 7, wherein the composition is configured to reduce wet combing force by at least about 90% compared to untreated hair.

9. The composition of claim 8, wherein the specific gravity is below about 0.88.

10. The composition of claim 9, wherein the composition provides a long-lasting smoothing effect by improving hair alignment and reducing frizz for at least about 24 hours after application.

11. The composition of claim 10, wherein the composition is free of parabens, sulfates, and non-biodegradable silicones.

12. The composition of claim 11, further comprising at least one secondary active ingredient selected from the group consisting of:
a Hyaluronic Acid Derivative, wherein the Hyaluronic Acid Derivative is selected from Sodium Hyaluronate, Hydroxypropyltrimonium Hyaluronate, or Hydrolyzed Sodium Hyaluronate, and is configured to enhance moisture retention and improve hair elasticity;
a conditioning agent, wherein the conditioning agent is selected from Amodimethicone, Dimethicone, or a cationic polymer, and is configured to smooth the hair cuticle, reduce friction, and improve manageability;
a structuring and stabilizing agent, wherein the structuring and stabilizing agent is selected from one or more of Cetearyl Alcohol, Cetyl Hydroxyethylcellulose, and Xanthan Gum, and is configured to maintain the aerated, whipped texture of the composition and ensure uniform application; and
wherein the secondary active ingredients work synergistically with the Hydrolyzed Vegetable Protein-Silicone Hybrid and the botanical antioxidant to improve the deposition, absorption, and performance of the rinse-off hair treatment composition while maintaining a lightweight, non-greasy feel.

13. The composition of claim 12, wherein the at least one secondary active ingredient is present in the following amounts by total weight of the composition:
a Hyaluronic Acid Derivative, selected from Sodium Hyaluronate, Hydroxypropyltrimonium Hyaluronate, or Hydrolyzed Sodium Hyaluronate, is present in an amount ranging from about 0.001% to about 0.50%;
a conditioning agent, selected from Amodimethicone, Dimethicone, or a cationic polymer, is present in an amount ranging from about 0.10% to about 5.00%;
a structuring and stabilizing agent, selected from one or more of Cetearyl Alcohol, Cetyl Hydroxyethylcellulose, and Xanthan Gum, is present in an amount ranging from about 0.10% to about 4.00%; and
wherein the selected secondary active ingredients, in the specified ranges, optimize the hydration, conditioning, and structural stability of the rinse-off hair treatment composition while maintaining its low-density, aerated texture and rinseability.

14. A rinse-off, low-specific-gravity, hair-repair treatment composition comprising:
about 0.175% by total weight of a Hydrolyzed Vegetable Protein-Silicone Hybrid, wherein the Hydrolyzed Vegetable Protein-Silicone Hybrid comprises Hydrolyzed Vegetable Protein PG-Propyl Silanetriol, and is configured to penetrate hair fibers to restore broken disulfide bonds, reinforce keratin structures, and improve tensile strength;
about 0.050% by total weight of a botanical antioxidant, wherein the botanical antioxidant comprises Silybum Marianum Seed Extract, and is configured to neutralize oxidative stress, protect restored disulfide bonds from degradation, and enhance overall hair resilience;
about 0.0025% by total weight of a Hyaluronic Acid Derivative, wherein the Hyaluronic Acid Derivative is Sodium Hyaluronate, and is configured to bind moisture to the hair shaft, improve hydration retention, and enhance elasticity;
about 0.00188% by total weight of a Cationic Hyaluronic Acid Derivative, wherein the Cationic Hyaluronic Acid Derivative is Hydroxypropyltrimonium Hyaluronate, and is configured to form a lightweight hydrating film on the hair surface, reduce friction, and improve detangling and smoothness;
about 0.00063% by total weight of a hydrolyzed low-molecular-weight Hyaluronic Acid Derivative, wherein the hydrolyzed low-molecular-weight Hyaluronic Acid Derivative is Hydrolyzed Sodium Hyaluronate, and is configured to penetrate into the hair cortex, deliver long-lasting hydration from within, and increase flexibility;
about 2.00% by total weight of a conditioning agent, wherein the conditioning agent comprises a combination of Amodimethicone and Dimethicone, and is configured to selectively deposit on damaged areas of the hair, create a flexible protective film, smooth the cuticle, and reduce frizz without excessive buildup;
about 1.50% by total weight of a structuring and stabilizing agent, wherein the structuring and stabilizing agent comprises a combination of two or more of Cetearyl Alcohol, Cetyl Hydroxyethylcellulose, and Xanthan Gum, and is configured to maintain an aerated, whipped texture, stabilize the formulation, and enhance uniform application;
water and additional cosmetically acceptable excipients, including pH adjusters, emulsifiers, preservatives, and rheology modifiers, wherein the rinse-off hair treatment composition has a specific gravity below about 0.90, forming a low-density, aerated, and easily spreadable formulation that enhances deposition and absorption while maintaining a lightweight, non-greasy feel;
wherein when the rinse-off hair treatment composition is applied to hair, left for at least 10 minutes, and rinsed, the rinse-off hair treatment composition restores at least about 32% of lost disulfide bonds, improves the visual appearance of at least about 68% of split ends, with at least about 46% of the split ends achieving complete closure, and reduces hair breakage by at least about 80% after 2,000 brush strokes in repeated grooming tests; and
wherein the synergistic combination of the Hydrolyzed Vegetable Protein-Silicone Hybrid, botanical antioxidant, Hyaluronic Acid Derivatives, and conditioning agents works to restore molecular hair integrity, reduce cortical fragmentation, enhance cuticle alignment, improve hydration retention, and provide long-lasting smoothness and frizz control, resulting in stronger, healthier, and more manageable hair.

15. A method for restoring disulfide bonds, reducing hair breakage, and improving the visual appearance of split ends in damaged hair, the method comprising:
applying a rinse-off hair treatment composition to damp hair, wherein the rinse-off hair treatment composition comprises:
a therapeutically effective amount of a Hydrolyzed Vegetable Protein-Silicone Hybrid, wherein the Hydrolyzed Vegetable Protein-Silicone Hybrid comprises Hydrolyzed Vegetable Protein PG-Propyl Silanetriol, and is configured to penetrate hair fibers to restore broken disulfide bonds, reinforce keratin structures, and improve tensile strength;
a therapeutically effective amount of a botanical antioxidant, wherein the botanical antioxidant comprises Silybum Marianum Seed Extract, and is configured to neutralize oxidative stress, protect restored disulfide bonds from degradation, and enhance overall hair resilience;
a therapeutically effective amount of a Hyaluronic Acid Derivative, wherein the Hyaluronic Acid Derivative is Sodium Hyaluronate, and is configured to bind moisture to the hair shaft, improve hydration retention, and enhance elasticity;
a therapeutically effective amount of a Cationic Hyaluronic Acid Derivative, wherein the Cationic Hyaluronic Acid Derivative is Hydroxypropyltrimonium Hyaluronate, and is configured to form a lightweight hydrating film on the hair surface, reduce friction, and improve detangling and smoothness;
a therapeutically effective amount of a hydrolyzed low-molecular-weight Hyaluronic Acid Derivative, wherein the hydrolyzed low-molecular-weight Hyaluronic Acid Derivative is Hydrolyzed Sodium Hyaluronate, and is configured to penetrate into the hair cortex, deliver long-lasting hydration from within, and increase flexibility;
a therapeutically effective amount of a conditioning agent, wherein the conditioning agent comprises a combination of Amodimethicone and Dimethicone, and is configured to selectively deposit on damaged areas of the hair, create a flexible protective film, smooth the cuticle, and reduce frizz without excessive buildup;
a therapeutically effective amount of a structuring and stabilizing agent, wherein the structuring and stabilizing agent comprises a combination of Cetearyl Alcohol, Cetyl Hydroxyethylcellulose, and Xanthan Gum, and is configured to maintain an aerated, whipped texture, stabilize the formulation, and enhance uniform application;
water and additional cosmetically acceptable excipients, including pH adjusters, emulsifiers, preservatives, and rheology modifiers;
wherein the rinse-off hair treatment composition has a specific gravity below about 0.88, forming a low-density, aerated, and easily spreadable formulation that enhances deposition and absorption while maintaining a lightweight, non-greasy feel;
massaging the rinse-off hair treatment composition into the hair fibers for at least 30 seconds, ensuring even distribution and penetration into the hair cortex;
leaving the rinse-off hair treatment composition on the hair for at least 10 minutes to allow sufficient absorption and molecular repair;
rinsing the rinse-off hair treatment composition from the hair with water at a controlled temperature, removing excess formulation while retaining the deposited conditioning agents;
wherein after a single application of the method, the rinse-off hair treatment composition restores at least about 30% of lost disulfide bonds, improves the visual appearance of at least about 60% of split ends, with at least about 40% of the split ends achieving complete closure, and reduces hair breakage by at least about 80% after 2,000 brush strokes in repeated grooming tests;
wherein the synergistic combination of the hydrolyzed vegetable protein-silicone hybrid, botanical antioxidant, hyaluronic acid derivatives, and conditioning agents works to restore molecular hair integrity, reduce cortical fragmentation, enhance cuticle alignment, improve hydration retention, and provide long-lasting smoothness and frizz control, resulting in stronger, healthier, and more manageable hair.

16. The method of claim 15, wherein the rinse-off hair treatment composition comprises:
a Hydrolyzed Vegetable Protein-Silicone Hybrid in an amount ranging from about 0.05% to about 2.00% by total weight of the composition;
a botanical antioxidant in an amount ranging from about 0.025% to about 1.00% by total weight of the composition;
a Hyaluronic Acid Derivative in an amount ranging from about 0.001% to about 0.50% by total weight of the composition;
a Cationic Hyaluronic Acid Derivative in an amount ranging from about 0.001% to about 0.30% by total weight of the composition;
a hydrolyzed low-molecular-weight Hyaluronic Acid Derivative in an amount ranging from about 0.0005% to about 0.20% by total weight of the composition;
a conditioning agent in an amount ranging from about 0.10% to about 5.00% by total weight of the composition;
a structuring and stabilizing agent in an amount ranging from about 0.10% to about 4.00% by total weight of the composition; and
wherein the composition retains a specific gravity below about 0.88 and maintains an aerated, whipped structure for optimal deposition and absorption into the hair fibers.

17. The method of claim 16, wherein the rinse-off hair treatment composition, when applied according to the method, reduces wet combing force by at least about 90% compared to untreated hair, facilitating improved detangling and hair manageability.

18. The method of claim 17, wherein after five consecutive applications, the rinse-off hair treatment composition restores at least about 70% of lost disulfide bonds, demonstrating cumulative structural reinforcement of the keratin network.

19. The method of claim 18, wherein the improvement in the visual appearance of split ends, including at least 40% achieving complete closure, is maintained for at least 72 hours post-treatment, resulting in long-lasting cuticle realignment and smoother hair texture.

20. The method of claim 19, wherein the rinse-off hair treatment composition reduces frizz for at least 24 hours after treatment and maintains hair alignment even under exposure to 80% relative humidity, demonstrating superior moisture resistance and cuticle smoothing effects.
